(19) United States
(12) Patent Application Publication
(10) Pub. No.: US 2018/0161626 A1
(43) Pub. Date: Jun. 14, 2018

(54) TARGETED NEUROGENESIS STIMULATED BY AEROBIC EXERCISE WITH BRAIN FUNCTION-SPECIFIC TASKS

(71) Applicant: BLUE GOJI LLC, Austin, TX (US)

(72) Inventor: Coleman Fung, Spicewood, TX (US)

(21) Appl. No.: 15/838,531

(22) Filed: Dec. 12, 2017

Related U.S. Application Data
(60) Provisional application No. 62/433,197, filed on Dec. 12, 2016.

(51) Int. Cl. A63B 24/00; A61B 5/16; A61B 5/22; A61B 5/02; G09B 5/02; G09B 23/28; A61B 5/00; G09B 7/02; H04L 29/08

(52) U.S. Cl. CPC A63B 24/0062; A61B 5/162; A61B 5/165; A61B 5/221; A61B 5/02; A63B 2024/0065; G09B 23/28; A61B 5/4884; G09B 7/02; H04L 67/10; A63B 2024/009; G09B 5/02

(57) ABSTRACT

A system targeted neurogenesis stimulated by aerobic exercise with brain function-specific tasks. A user may participate in game-like tasks that challenge specific brain functions has been developed. This system comprises a central game server and an aerobic exercise machine. The central game server acts as a source of games specifically designed to repetitively challenge one of more brain functions while maintaining maximal user attention. The aerobic exercise machine promotes engagement in an exercise plan and monitors physiological data to maintain level of intensity within pre-programmed range.

### CROSS-REFERENCE TO RELATED APPLICATIONS

This application claims priority to U.S. provisional patent application Ser. No. 62/433,197, titled “TARGETED NEUROGENESIS STIMULATED BY AEROBIC EXERCISE WITH BRAIN FUNCTION-SPECIFIC TASKS”, and filed on Dec. 12, 2016, the entire specification of which is incorporated herein by reference.

### BACKGROUND OF THE INVENTION

### Field of the Art

The disclosure relates to the fields of aerobic exercise-enhanced neurogenesis and the use of tasks that challenge specific brain regions to improve or to maintain a group of brain functions, and more particularly to the field of use of video and virtual reality games in conjunction with managed aerobic exercise to drive enhanced functions in targeted regions of the brain.

### Discussion of the State of the Art

The discovery that neurogenesis occurs in at least one structure of adult human brains has recently driven a great deal of research into this biological process. Of great interest are findings that engaging in regular, moderate, aerobic exercise is a major driver of neurogenesis in the hippocampus specifically the dentate gyrus, and the amygdala. Further, in addition to neurogenesis, it was determined that the new neurons and the improved connections fully differentiate and appear to function within these structures, this effect has been found to occur in old as well as young test subjects, indicating that in people of all ages the central nervous system retains a level of plasticity and that a known activity that can be performed by most can positively influence continued brain health. Especially encouraging are the findings that aerobic exercise and the resultant neurogenesis has demonstrable positive effects on such neurological diseases as Alzheimer's Disease where moderate aerobic exercise significantly prolonged elevated learning, cognitive, and memory abilities of early diagnosed patients; post-traumatic stress disorder where sufferers placed on aerobic exercise programs showed significantly reduced flashbacks to past events, better differentiation of past events where danger was significant and of later trigger events that may extend to the present where minimal of no danger existed; major depression, where exercise, specifically aerobic exercise has long been suspected to reduce severity, and where neurogenesis is now at least tightly correlated with symptom improvement; and some higher functioning autism spectrum disorder cases where sufferers placed on an aerobic exercise program showed better social cue understanding and social interaction. A study done on random children showed that, even when no detectable deficits are present, aerobic exercise programs increased learning potential, and that when other factors are separated out, these improvements can be attributed to neurogenesis. These are just some examples of improvement attributed to neurogenesis in hippocampus and amygdala. More recent studies have indicated that aerobic exercise also drives at least some neurogenesis—both new neurons and their connections—in other areas of the brain such as the pre-frontal, frontal and temporal lobes of the cortex, and early studies further indicate that neurogenesis in these regions lead to functional improvements in tasks controlled by these regions, both when deficits were present and when no detectable deficit was found.

The mechanism of increased neurogenesis in the above examples has been traced through animal studies and analysis of peripheral blood levels in humans to the effects of growth factors which include brain-derived neurotrophic factor, nerve growth factor, and insulin-like growth factor-1. Moderate to high intensity aerobic exercise was found to promote the highest levels of these and other growth factors in the brain while other forms of exercise such as weight training or interval type training which produces more stress on the body and over shorter time intervals, produced much less production of the growth factors and are reported to produce significantly lesser functional brain effects when effects were seen. These data indicates that aerobic exercise has characteristics uniquely suited to producing neurogenesis.

Another area of recent and productive brain research is the use of specific, targeted tasks to challenge specific functional areas of the brain to produce increased proficiency in those functions. Much of the current research pertains to the neurophysiological area of executive function which is comprised of underlying processes of mental set shifting, information updating, and inhibition or prepotent responses, which when combined allow humans to make decisions and form plans of action rapidly in the presence of complex, incomplete and sometimes conflicting data. Research has shown that presenting test subjects with repetitive changing tasks which isolate and challenge these processes improves proficiency in executive function as a whole and that this improvement extends to all of the wide range of varied tasks which use executive function capabilities not just those tasks specifically challenged. Generalization of the trained proficiency from the trained tasks to tasks requiring similar brain function is a novel development and further research is beginning to generalize this finding to other functional areas of the brain. Such studies have suggested that repetitive challenges to the function of interest and maintenance of high levels of attention, interest, and participation throughout the process results in the most substantial gains. To this end, multiple researchers now employ video games into which tasks designed to challenge a specific brain function or related brain functions as a means of fulfilling these findings are incorporated. This use of video games with incorporated targeted brain challenges has proven a potent tool in generalizable training of specific brain functions.

What is needed, is a system and method that combines the neurogenesis-producing capability of aerobic exercise and the potency of well-designed, task-specific video games and particularly with immersive virtual or augmented reality games, to promote increased or to maintain proficiency in targeted brain functions using the synchronized effects of targeted stimulation and aerobic exercise-induced neurogenesis.

### SUMMARY OF THE INVENTION

Accordingly, the inventor has conceived and reduced to practice, in a preferred embodiment of the invention, a system and method for targeted and improved neurogenesis stimulated by aerobic exercise with brain function-specific tasks, during which a user may participate in game-like mental tasks that challenge specific brain functions while exercising in sync with such tasks with the end result being enhanced brain function, that promotes increased or maintains proficiency and/or recovery of damaged competence in targeted brain functions using the combined, synchronized effects of targeted brain function stimulation and aerobic exercise-induced neurogenesis. The targeted brain functions include, but not limit to, cognitive skills, motor skills, memory functions, focus and concentration skills, emotional/empathic responses, social/collaboration skills, compulsive/addictive behaviors, and anxiety attacks.

The problem of combining the neurogenesis-producing potential of aerobic exercise with the potency of video games and particularly with immersive virtual or augmented reality games, to promote increased or to maintain proficiency and/or recovery of damaged competence in targeted brain functions using the combined, synchronized effects of targeted stimulation and aerobic exercise-induced neurogenesis, is solved by using exercise equipment such as elliptical trainers and stationary bikes as well as optional physiological data monitoring devices to promote habitual long-term exercise programs of preprogrammed optimal intensity for maximal brain cell generation as has been reported to be an effect of aerobic exercise based on a user's age and physical conditions. Also part of the system of the invention is a video game environment with one or more of the following accessories: virtual reality or augmented reality headsets, handheld or exercise equipment mounted controllers of varying configurations, each meant to serve optimally for specific types of game function, a biometrics headset designed to monitor electrical brain activity (EEG), muscle activity (EMG), and heart rate (EKG), and other bio-electrical activities, a tethered harness designed to allow a user's body position to control video game play as well as other controllers or sensors which may support limited physical motions due to disability. Recent research has determined that the use of video games to repetitively challenge functions of the brain can lead to generalizable improvement in those functions when properly presented during play sessions. Video games in this system are specifically designed to promote immersive levels of attention and to incorporate tasks which challenge specific brain functions whether they be cognitive for general individual enhancement; or as would be encountered in cases of dementia including Alzheimer's Disease, and higher functioning autism spectrum disorder among others; or due to accident such as traumatic brain injuries; or to enhance physical ability such as fine motor coordination, again either for individual improvement of sub-average ability or to recover ability due to developing or newly formed physical disabilities such as Parkinson's disease, stroke or traumatic brain injury. Towards this goal, the system tightly integrates exercise and immersive game play to maximize adherence to aerobic exercise plan, and to expose the user to multiple repetitions of the immersive brain function stimulating games, both of which have been found crucial to the success of the two techniques, aerobic exercise and task specific training of the brain separately.

According to a preferred embodiment of the invention, a system for targeted neurogenesis stimulated by aerobic exercise with brain function-specific tasks, comprising: a game server operating on a computing device comprising at least a processor and a memory and configured to: operate a game-oriented software program comprising game data configured to stimulate at least a specific brain function in a user during gameplay; transmit at least a portion of the game data to a virtual reality device; receive user input from a plurality of devices via a network; and direct the operation of the game-oriented software program based at least in part on the user input; a virtual reality device comprising at least a processor and a memory and a plurality of hardware sensors and a display device, and configured to: receive game data from the game server; display at least a portion of the game data to a user using the display device; transmit operating instructions to a plurality of external devices, the instructions being based at least in part on at least a portion of the game data; and receive input from the user via at least a portion of the plurality of hardware sensors; and transmit at least a portion of the user input to the game server, is disclosed.

According to another preferred embodiment of the invention, a method for targeted neurogenesis stimulated by aerobic exercise with brain function-specific tasks, comprising the steps of: operating, using a game server operating on a computing device comprising at least a processor and a memory, a game-oriented software program comprising game data configured to stimulate at least a specific brain function in a user during gameplay; transmitting at least a portion of the game data to a virtual reality device; receiving, at a virtual reality device comprising at least a processor and a memory and a plurality of hardware sensors and a display device, game data from the game server; displaying at least a portion of the game data to a user using the display device; receiving input from the user via at least a portion of the plurality of hardware sensors; transmitting at least a portion of the user input to the game server; and directing the operation of the game-oriented software program based at least in part on the user input, is disclosed.

According to an additional embodiment of the invention, the system for targeted neurogenesis stimulated by aerobic exercise with brain function-specific tasks, wherein the game server is further configured to: receive biometric data from a plurality of fitness devices; and direct the operation of the game-oriented software program based at least in part on the received biometric data, is disclosed.

According to an additional embodiment of the invention, the method for targeted neurogenesis stimulated by aerobic exercise with brain function-specific tasks, further comprising the steps of: receiving biometric data from a plurality of fitness devices; and directing the operation of the game-oriented software program based at least in part on the received biometric data, is disclosed.

According to further embodiments of the system and method described herein, the plurality of fitness data may comprise heart rate data, blood oxygenation data, or accelerometer data.

### BRIEF DESCRIPTION OF THE DRAWING FIGURES

The accompanying drawings illustrate several embodiments of the invention and, together with the description, serve to explain the principles of the invention according to the embodiments. It will be appreciated by one skilled in the art that the particular embodiments illustrated in the drawings are merely exemplary, and are not to be considered as limiting of the scope of the invention or the claims herein in any way.

FIG. 1 is a block diagram of an exemplary system architecture for targeted neurogenesis augmented by aerobic exercise, according to a preferred embodiment of the invention.

FIG. 2 is a flow diagram illustrating an exemplary method of operation of a system for targeted neurogenesis augmented by aerobic exercise according to a preferred embodiment of the invention.

FIG. 3A is a flow diagram illustrating an exemplary method of operation of a system for targeted neurogenesis induced by aerobic exercise and brain function specific tasks or game plays according to an alternate embodiment of the invention.

FIG. 3B is a flow diagram illustrating an exemplary method of operation of a system for targeted neurogenesis induced by aerobic exercise and brain function specific tasks or game plays according to an alternate embodiment of the invention.

FIG. 4 is a block diagram of an exemplary virtual reality/augmented reality viewer according to a preferred embodiment of the invention.

FIG. 5 comprises diagrams illustrating exemplary hand operated controllers, according to a preferred embodiment of the invention.

FIG. 6A is a diagram illustrating aspects of an exemplary foot operated controller, according to a preferred embodiment of the invention.

FIG. 6B is a diagram illustrating aspects of an exemplary foot operated controller, according to a preferred embodiment of the invention.

FIG. 6C is a diagram illustrating aspects of an exemplary foot operated controller, according to a preferred embodiment of the invention.

FIG. 7 comprises diagrams illustrating aspects of an exemplary arrangement of a system for targeted neurogenesis augmented by aerobic exercise, illustrating the use of fixed controllers and body positioning on a treadmill, as well as additional embodiment features of the treadmill according to a preferred embodiment of the invention.

FIG. 8 is a diagram of an exemplary hardware arrangement of an apparatus for natural torso stabilization, tracking and feedback for electronic interaction illustrating the use of multiple tethers and a movable torso harness.

FIG. 9A is a diagram illustrating physiological monitoring devices according to a preferred embodiment of the invention.

FIG. 9B is another diagram illustrating physiological monitoring devices according to a preferred embodiment of the invention.

FIG. 10 is a flow diagram illustrating the use of brain monitoring equipment to evaluate efficacy of game-like targeted brain task programming according to a preferred embodiment of the invention.

FIG. 11A is a simplified rendering illustration showing memory enhancing tasks presented in an aerobic-game format according to an embodiment of the invention.

FIG. 11B is a simplified rendering illustration showing memory enhancing tasks presented in an aerobic-game format according to an embodiment of the invention.

FIG. 11C is an illustration of exemplary questions that may be asked after completing a memory enhancing task according to an embodiment of the invention.

FIG. 12 is a set of simplified illustrations showing a generalized aerobic exercise brain function enhancement game which may be used for multiple brain function enhancement tasks.

FIG. 13 is a block diagram illustrating an exemplary hardware architecture of a computing device used in an embodiment of the invention.

FIG. 14 is a block diagram illustrating an exemplary logical architecture for a client device, according to an embodiment of the invention.

FIG. 15 is a block diagram showing an exemplary architectural arrangement of clients, servers, and external services, according to an embodiment of the invention.

FIG. 16 is another block diagram illustrating an exemplary hardware architecture of a computing device used in various embodiments of the invention.

### DETAILED DESCRIPTION

The inventor has conceived, and reduced to practice, in a preferred embodiment of the invention, a system and method for targeted neurogenesis stimulated by aerobic exercise with brain function-specific tasks.

One or more different inventions may be described in the present application. Further, for one or more of the inventions described herein, numerous alternative embodiments may be described; it should be appreciated that these are presented for illustrative purposes only and are not limiting of the inventions contained herein or the claims presented herein in any way. One or more of the inventions may be widely applicable to numerous embodiments, as may be readily apparent from the disclosure. In general, embodiments are described in sufficient detail to enable those skilled in the art to practice one or more of the inventions, and it should be appreciated that other embodiments may be utilized and that structural, logical, software, electrical and other changes may be made without departing from the scope of the particular inventions. Accordingly, one skilled in the art will recognize that one or more of the inventions may be practiced with various modifications and alterations. Particular features of one or more of the inventions described herein may be described with reference to one or more particular embodiments or figures that form a part of the present disclosure, and in which are shown, by way of illustration, specific embodiments of one or more of the inventions. It should be appreciated, however, that such features are not limited to usage in the one or more particular embodiments or figures with reference to which they are described. The present disclosure is neither a literal description of all embodiments of one or more of the inventions nor a listing of features of one or more of the inventions that must be present in all embodiments.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an embodiment with several components in communication with each other does not imply that all such components are required. To the contrary, a variety of optional components may be described to illustrate a wide variety of possible embodiments of one or more of the inventions and in order to more fully illustrate one or more aspects of the inventions. Similarly, although process steps, method steps, algorithms or the like may be described in a sequential order, such processes, methods and algorithms may generally be configured to work in alternate or random orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the invention(s), and does not imply that the illustrated process is preferred. Also, steps are generally described once per embodiment, but this does not mean they must occur once, or that they may only occur once each time a process, method, or algorithm is carried out or executed. Some steps may be omitted in some embodiments or some occurrences, or some steps may be executed more than once in a given embodiment or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments of one or more of the inventions need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of embodiments of the present invention in which, for example, functions may be executed out of order from that shown or discussed, including substantially concurrently or in reverse order, depending on the functionality involved, as would be understood by those having ordinary skill in the art.

### Conceptual Architecture

### Detailed Description of Exemplary Embodiments

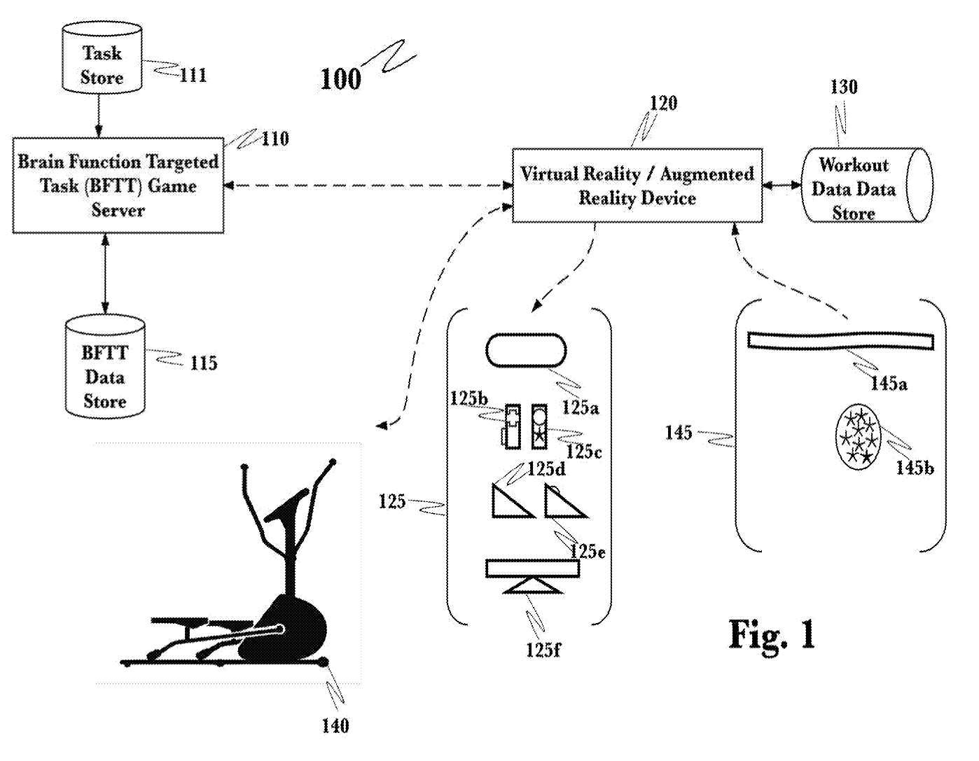

FIG. 1 is a block diagram of an exemplary system architecture for targeted neurogenesis augmented by aerobic exercise 100, according to a preferred embodiment of the invention. Important characteristics of a program meant to target and enhance one or more specific brain functions, each often resident in a specific brain region, is to focus as much participant attention as possible, ideally total attention, towards the task being presented for periods of time that may extend many minutes in duration. Such attention capture may require embedding the task into game-like settings where the participant may be presented with stimulating action, competitive situations, or immersive role playing, among other circumstances known to both capture and retain attention focus known to those skilled in the art, which involve accomplishing the targeted task. For example, a participant working on short term memory may be given a series of icons or small images to remember at the start of a virtual reality ski race and then have to quickly select them on the course in changed order or know what position in the series a specific icon or image occupied in order to attain the fastest path down the hill and win the race. Additionally, the participant's detailed action, response, answer, or result to each game play or task may be captured and analyzed over time to monitor the participant's overall progress for a targeted brain function. This progress feedback may in turn drive or influence new iterations of game plays or tasks for future play or practice sessions. Multiple studies have confirmed that aerobic exercise leads to neurogenesis and improved function within at least certain structures within the brain. Aerobic exercise then may augment the gains made by performing brain function targeted tasks.

An embodiment of a system to combine both aerobic exercise and brain function or brain region developing tasks 100 may comprise a centralized brain function targeted task (BFTT) game server 110, which acts as a source for game programming and providing tasks or gameplay elements for use. Those programs may be kept in a permanent task store 111 present for that purpose. A BFTT data store 115 may also be present, especially during development of game programming to challenge novel brain functions, to permanently keep efficacy data monitored during participant virtual reality/augmented reality experience 120 through the use of one or more biometric devices 145 for later analysis. A workout data store 130 may also be used to store and provide workout-specific data for later analysis as it is collected during use. When efficacy is shown though test monitoring, 2D rendered puzzles or games may also be used by some embodiments in this setting. BFTT data store 115 data store may also permanently store certain return data from participant devices on an ongoing basis to monitor game appeal, usage and task improvement over time. Games challenging specific brain functions of interest to the customer either personally or as part of a physician directed regimen can be downloaded either wirelessly or using applicable wired standards by participant local virtual reality/augmented reality controllers 125 such as but not limited to a virtual reality/augmented reality viewer enclosed smartphone 125a or other computer device capable of presenting virtual reality/augmented reality content, examples of which are described below. These alternative consoles may also have an amount of session storage to allow for local graphing and other analytical review of both just completed sessions, analysis of individual previous sessions or comparative analysis of one or more parameters from a desired number of previous sessions on the part of the user or an administering professional. Such a system may make use of one or more of a plurality of wireless controllers, (for example, using Wi-Fi, BLUETOOTH™, or ANT™ protocols), some of which may be hand held and operated 125b, 125c, these handheld controllers possibly having different capabilities such as but not limited to top facing buttons 125b, 125c, side facing buttons 125b, accelerometers, and haptic feedback generation, among other capabilities known to those skilled in the art as needed to play the targeted task games. Other controllers may be activated by all or portions of a participant's foot 125d, 125e, possibly with further input mediated by toe activated buttons 125e, while still others may have one or more of several other modes such as monitoring balance or use of intentional uneven foot pressure as might occur while using a skate park or traversing portions of a snowboarding course 125f. The invention may be configured interact with any other controller types known to those skilled in the art as needed.

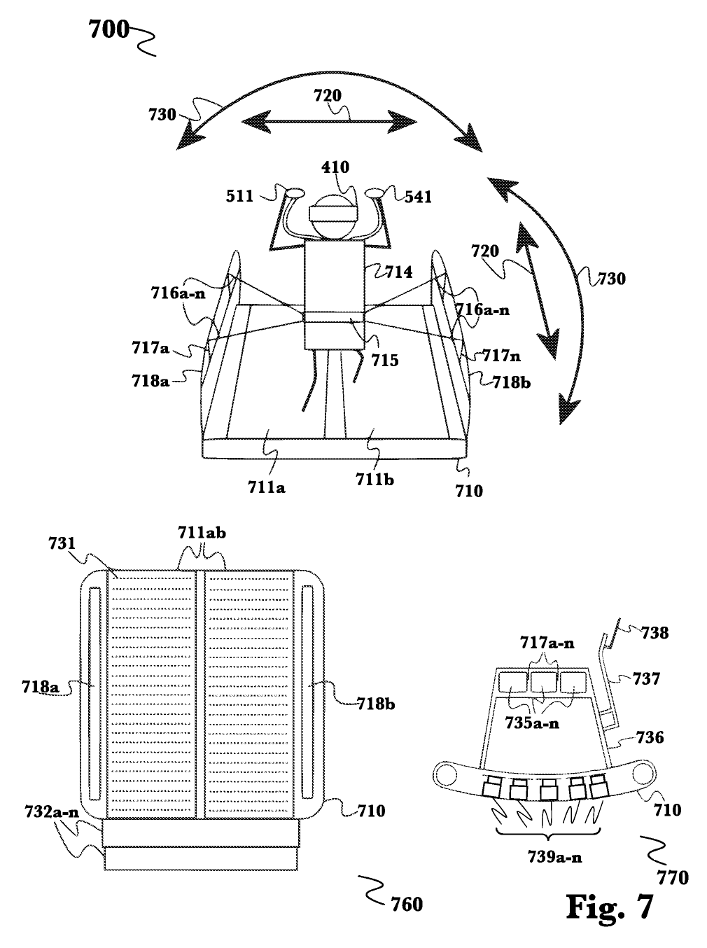

While different embodiments may use different types of aerobic exercise equipment such as but not limited to elliptical 140, exercise bike (not shown), VR-specific, enhanced treadmill (see FIG. 7), rowing machine (not shown) and cross-country ski machine, just to name a few examples known to those skilled in the art, some type of aerobic exercise is part of the system. Under the embodiment, aerobic exercise equipment 140 may be integrated with an embodiment and thus communicate the virtual reality/augmented reality controller; or, the aerobic equipment used may be independent of the brain-function-targeted-task game server based upon pre-existing ownership or participant preference.

Different studies of the effects of aerobic exercise on neurogenesis as it relates to specific deficiencies such as depression, dementia, and autism spectrum disorders as well as enhancement of abilities such as learning and executive function, among other desirable outcomes known to those skilled in the art, specify a range of activity level such as light aerobic activity, medium aerobic activity, and intense aerobic activity, as ideal for the desired neurogenic functional effect. Taken together, the plurality of studies may present evidence that different levels of aerobic intensity may be ideal for specific aerobic exercise mediated functional outcomes. The system may thus make use of a heart-rate and respiration sensor band 145a to regulate a participant' s exercise intensity level to be within the range specified for the desired neurogenically mediated functional outcome. Alternatively, fitness trackers (for example including, but not limited to, FITBIT™, MICROSOFT BAND™, or APPLE WATCH™ devices) may be paired via a wireless personal-area-network protocol such as BLUETOOTH™ or ANT™, to provide a user's biometric or health information. Feedback may also be provided at times by other physiological monitoring devices, one such being measurement of brain activity as may be gathered using a EEK monitoring headset 145b during the aerobic exercise-task performance session to confirm that the correct patterns of activity occur for the desired outcome. This type of monitoring may be especially important during programming of the game-like vehicles in which tasks to stimulate specific brain activities are embedded. Other physiological feedback not listed, for example a participant's blood pressure, glucose level or samples for determination of blood levels of certain hormones, but known to those skilled in the art may be gathered as appropriate to the circumstance.

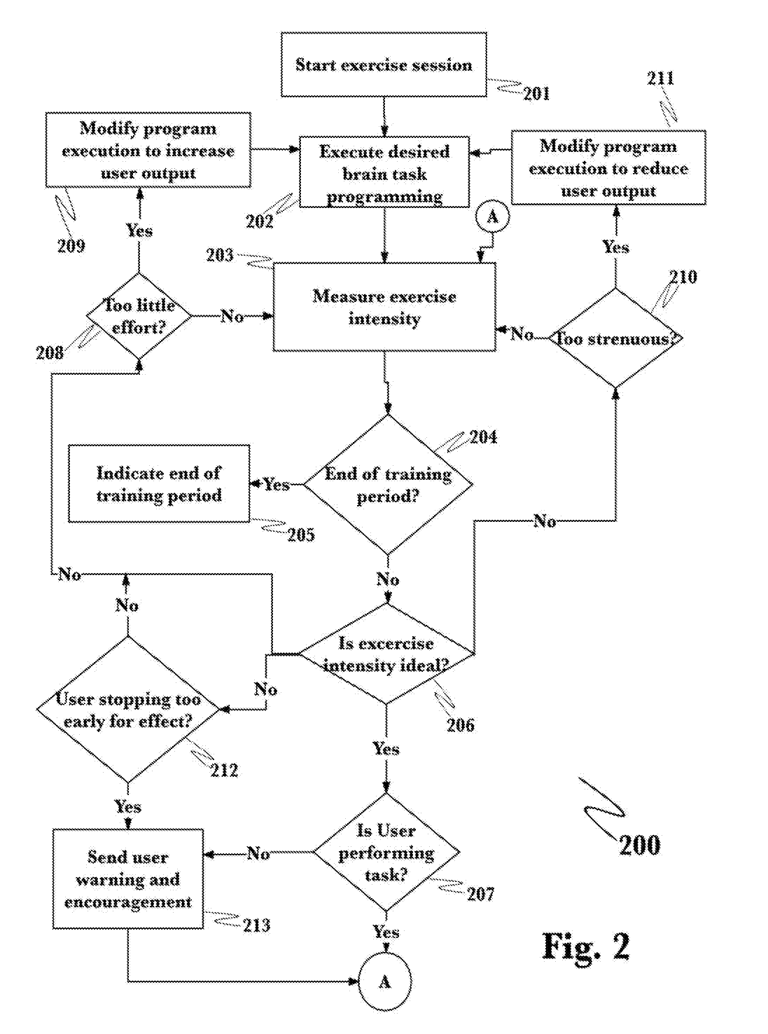

FIG. 2 is a flow diagram illustrating an exemplary method of operation of a system for targeted neurogenesis augmented by aerobic exercise 200 according to a preferred embodiment of the invention. Under one embodiment, aerobic exercise 201 and performance of targeted brain task including games 202 occur concurrently. Scientific investigation of multiple changes in brain function attributed to exercise augmented neurogenesis imply that different intensities of aerobic exercise such as “light intensity,” “medium intensity,” and “high intensity” give rise to optimal neurogenesis for desired outcome. To insure optimal neurogenesis mediated improvements in targeted brain function in the presence of a known desired outcome, the exertion level of the user, as measured by such parameters as heart and respiration rates may be monitored 203, 206 and the exertion required to play or the rate of activity occurring within in the virtual or augmented reality game may be reduced 211 to reduce user exertion when activity is determined too strenuous 210 on the other hand, the amount of exertion required to successfully play, or the rate of activity occurring in the virtual or augmented reality game may be increased 209 to cause exertion by the user to increase in cases where user effort is below optimal levels 208, or an indicator may be provided 205 that the session will end, if a configured training period has ended 204. The user may also become discouraged or distracted and stop exercise effort prior to the minimum time felt needed for desired effect 212. Under these conditions, the user may be shown a warning to that effect and may also be given some extra incentive or encouragement to continue the planned session 213. In the event that the user stops performing the assigned brain function targeted task prior to the minimal time period for efficacy 207, the user may encounter a warning and may further receive incentive such as flash effects, an opportunity to perform an heroic deed or an especially onerous character to further pursue to encourage continued participation 213. It is important to keep in mind that while the methods for the exercise component of the invention and the specific brain function stimulating puzzle solving or gameplay of the invention are presented separately here for clarity, and may be separated by some time interval in a small number of embodiments such as but not limited to under conditions of certain types of rehabilitation where physical disability may prevent concurrency, both neurogenesis inducing exercise and gameplay would occur concurrently under the majority of embodiments.

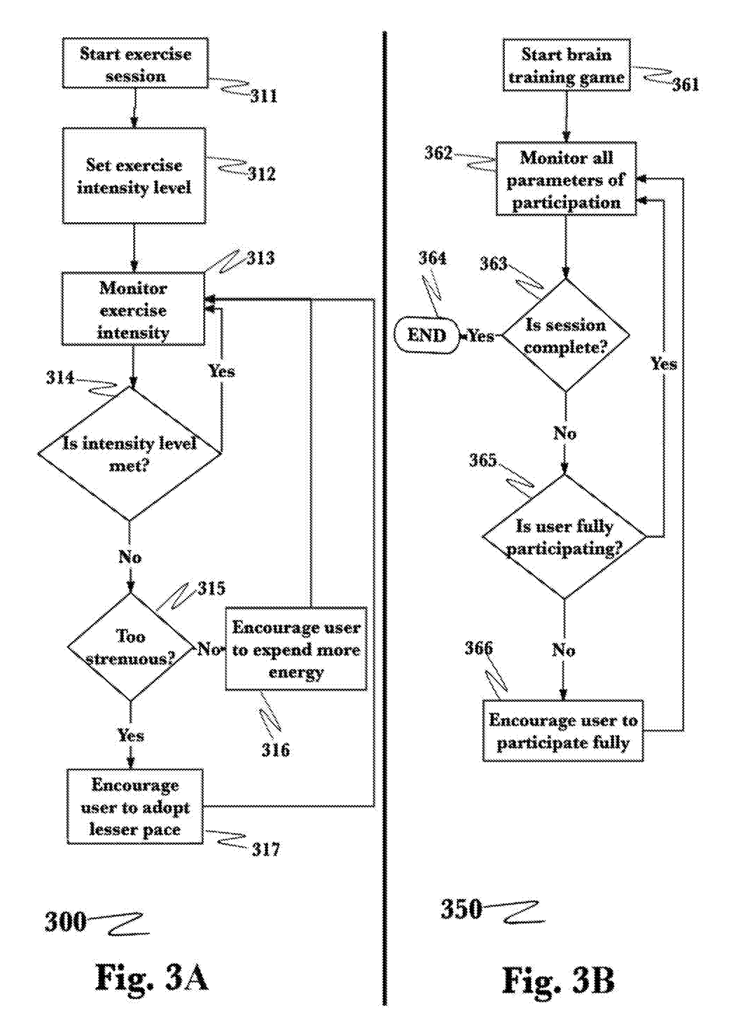

FIG. 3A is a flow diagram illustrating an exemplary method 300 of operation of a system for targeted neurogenesis induced by aerobic exercise and brain function specific tasks or game plays according to an alternate embodiment of the invention. According to the embodiment, users perform the prescribed exercise session 311 at a predetermined intensity level found optimal for task specific neurogenesis 312 which is then monitored 313 by the system 120 using physiological feedback sensors 145a. preprogrammed levels of deviation from the set exercise intensity levels 314 will result in alerts to the user to either expend less effort 315, 317 or expend more effort 316 to restore the optimal level.

FIG. 3B is a flow diagram illustrating an exemplary method 350 of operation of a system for targeted neurogenesis induced by aerobic exercise and brain function specific tasks or game plays according to an alternate embodiment of the invention. According to the embodiment, a user may start to play the virtual reality or augmented reality brain training games 361, the attention and engagement of the user towards the games is monitored throughout the session 362, optionally as well as user responses to prompt, actions performed, and other participation within the game. If, at some point in the session the user's attention, as measured by game guided user input, wanes below a preprogrammed level 365, the system may introduce more exciting play or graphical elements or may issue some other form of feedback, including haptic feedback, to re-engage that user 366. This set of activities continues until the session completes 363 at which time the game is brought to a satisfying end for the user 364. Additionally, participation may be used to drive gameplay modification 366 or collected for future use such as (for example) to analyze user performance or correlate user behavior with neurogenesis results.

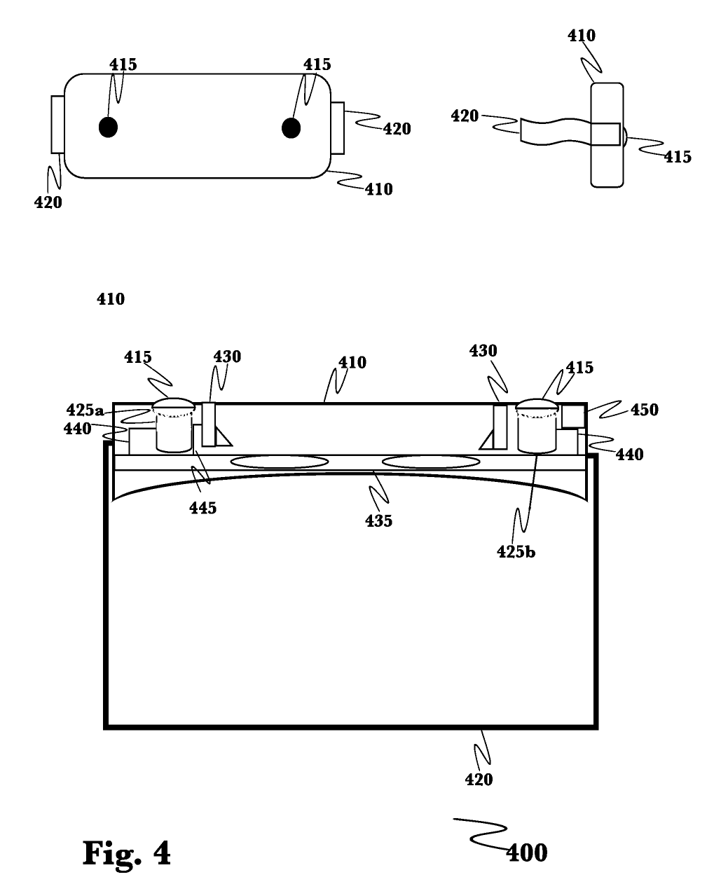

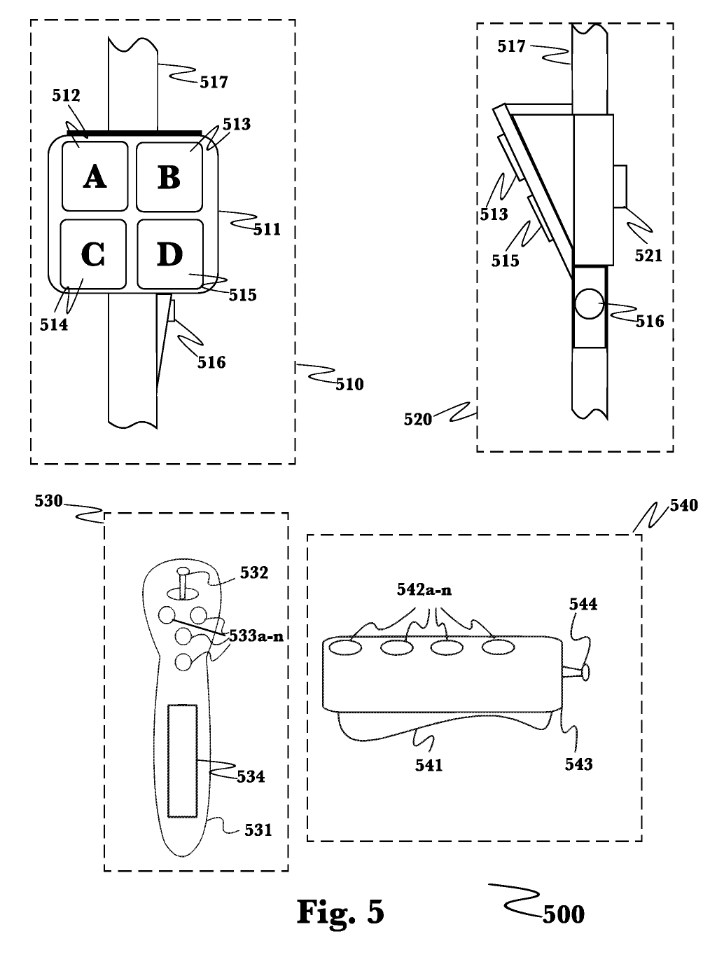

FIG. 4 is a block diagram of an exemplary virtual reality/augmented reality VR/AR headset 400 according to a preferred embodiment of the invention. Presentation of the virtual and augmented reality games into which brain function targeted tasks have been is mediated by a virtual reality or augmented reality headset 410. This headset may be wireless, possibly using WIFI™, BLUETOOTH™, or ANT™ communication protocols mediated by appropriate wireless radio 450. Wireless is the preferred headset configuration as it may allow for unrestricted movement during game play. To support augmented reality, the headset may have one or two HD video cameras 425a, 425b incorporated, the use of two cameras allowing full stereoscopic representation. In addition to the sensor 425a, 425b each camera comprises a focusing lens 415 and is mounted so as to approximate normal stereographic parallax. The headset may also include mounting brackets 430 for one or more VR capable smartphone models with a logic enabled connector 445 that allows the headset to act not only as a brain function targeted game media presentation device but as a game control device within the system 120. Some embodiments may include wireless VR/AR headsets specifically designed for the system in which case a dedicated controller computing device will be present in addition to a display screen. Headset may include close focal length lenses 435 to allow the user to focus on the display screen, retention system to keep the headset securely positioned on the user's head 420 and the needed communications busses for operation 440. Alternate embodiments may employ existing commercial VR gaming consoles such as, but not limited to Sony's PLAYSTATION VR™ console equipped with a VR headset, an OCULUS RIFT™ console with VR headset, or Microsoft's new version XBOX ONE™ VR console with a VR headset as brain function targeted task game devices 120.

FIG. 5 comprises diagrams illustrating a variety of exemplary physical configurations of nested-communication hand operated controllers 500, according to a preferred embodiment of the invention. According to one exemplary arrangement, a controller 511 may be configured for optimum use for response to games such as mazes and puzzles where multiple choices may be presented to the user. According to such an arrangement, a number of buttons 512, 513, 514, 515, 516, 521 may be arranged for use in navigating an on-screen interface where one or more choices or finger press sequences are made to progress in the game among other uses known to those skilled in the art. The button configuration may include a side facing button 516 positioned for access by the forefinger when the controller is mounted on a vertical exercise equipment pole 517 or by the thumb when mounted horizontally on a pole or railing. As illustrated in view 520, the forward facing buttons are mounted at an angle, to position them for easy and comfortable access during use, especially when the side facing button is used. The inclusion of four buttons and the labels on them were arbitrarily chosen as manageable in use and straightforward for presentation. Other numbers of buttons and button labels may be used within the design of the system. Button labels may be electronically or mechanically controlled and so may also change dependent on desired purpose. Additional or alternate controllers, features, or arrangements may also be utilized in various combinations or substitutions, for example to accommodate specific user needs such as physical ability or familiarity with a particular control scheme (such as for users with mental or physical disabilities that may affect their performance without accommodation).

According to another exemplary arrangement, a controller 530 may be configured for a particular gaming use, for example formed with an appropriate physical shape 531 and having control elements (buttons, switch, and other interactive hardware elements) arranged to best suit a simulation-type brain function targeted game. According to such a configuration, a control device 530 may have a digital or analog “joystick”-type control element 532 for use in controlling a player's character or vehicle in a 360-degree three-dimensional virtual environment, for example for use in third-person action or adventure-type games. A plurality of additional buttons 533a-n may be provided and arranged for easy one-handed use, such as to access a player's inventory, perform specific in-game actions (for example to jump, run, roll, or other movement controls), or to perform context-sensitive actions according to the nature of a particular game or application. For example, a simulator-oriented control device 530 may be paired with a controller device similar to 510 as a secondary controller, enabling a user to choose alternative answers to in-game queries or enter previously memorized codes or finger sequences and then use the second control device 530 to continue with other portions of the game. Controller 530 may also be equipped with a central pressure sensor in the handle 534 to allow certain users to perform brain function tasks involving grip it may be equipped with one or more accelerometers to measure orientation and controller movement for use in a plurality of game tasks including such game mediated tasks as tremor reduction or extinction for Parkinson's Disease patients, hand control re-establishment in stroke patients as well as other brain function tasks known to those skilled in the art.

According to a further exemplary arrangement, a control device may be configured for use specifically as a secondary controller, for example as a throttle controller 540 configured to be paired with a simulator controller 530 for use in a two-handed “hands on throttle and stick” (HOTAS) setup commonly used in flight-simulation games. According to such a configuration, a control device 540 may comprise a joystick or hat-switch control element 544 for navigation or for controlling a number of game or application functions with a user's thumb, generally positioned such that while holding control device 540 a user's thumb is naturally aligned with the control element 544 for ease of use. Control device 540 may further comprise an integrally-formed or removably affixed ergonomic portion 541, for example a removable or positionable rigid or soft panel, generally configured to conform to a user's hand or palm and align the user's hand for proper manipulation of various control elements. For example, in one exemplary arrangement, a control device 540 may be configured with a body 543 made of a rigid plastic material and having a removable plastic panel 541 shaped to fit a user's palm for a proper grip and alignment during use. Such a configuration may also be designed to accept a variety of alternate rigid panels 541, enabling a user to swap grip panels to configure the physical shape of control device 540 to most comfortably fit their particular anatomy (for example, such a configuration has been used in the firearms industry to enable users to configure the grip of a handgun for proper posture and aiming). Control device 540 may further comprise a plurality of additional control elements 542a-n such as buttons or switches, for example arranged such that when properly aligned (as by using an ergonomic grip panel, as described previously) a user's fingers may naturally be aligned in a position for ideal activation of control elements 542a-n. In this manner, it may be appreciated that a control device 510, 520, 530, 540 may be configured with ergonomic portions 541 in a variety of arrangements, and with various arrangements of control elements for optimal use when a user's hand is properly aligned and grasping control device 510, 520, 530, 540, such that any particular control device 510, 520, 530, 540 may be tailored to fit a wide range of users' anatomies for optimal use, as well as to promote ergonomic comfort and physical health. For example, by encouraging users to find an optimum ergonomic fit, the risk of repetitive-stress injuries and other health concerns may be reduced.

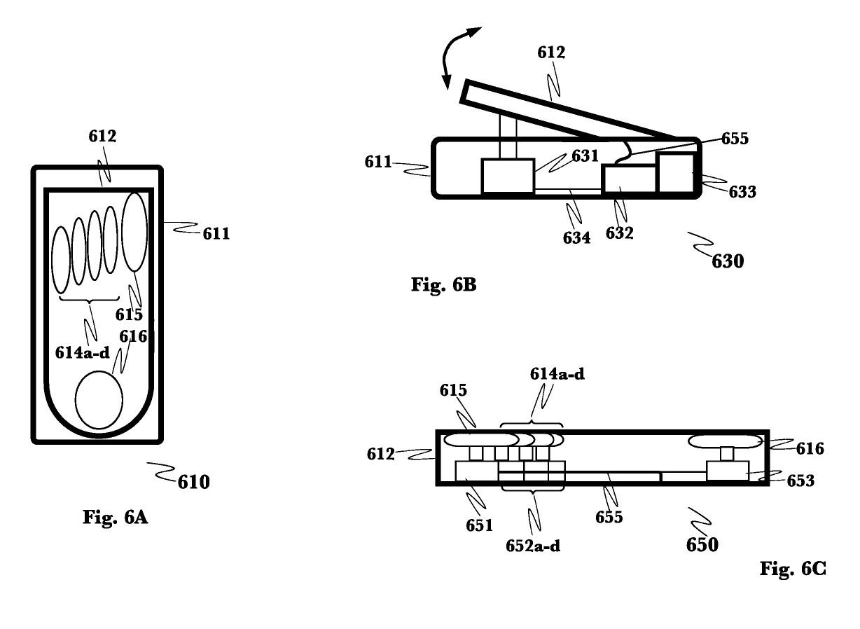

FIG. 6A is a diagram illustrating aspects of an exemplary foot operated controller 600, according to a preferred embodiment of the invention. The long standing success of repetitive use of appendages affected by medical insult such as stroke, spinal cord and traumatic brain injury among others familiar to those skilled in the art, when combined with evidence that aerobic exercise may promote neurogenesis in regions of the brain associated with motor function, as well as existence of those who have lost use of their hands, but who would greatly benefit from inclusion in programs to enhance specific brain functions through function targeted game play underscore the use of foot activated controllers 600, at least in a rehabilitation setting if not in the general population. One embodiment of a foot activated controller is depicted in

FIGS. 6A-C. Here 610 a foot activated pedal 612 is contained in a rigid enclosure 611. The pedal 612 pivots towards its base as depression of its distal end, corresponding to the toes of the foot, increases. The distal end also contains buttons corresponding to the toes of the foot 614a-d, 615 that can be configured for function depending on the abilities of the user and the role desired. Often the individual smaller toe buttons 614a-d may be configured as a single activator and the big toe button configured as a second individual activator unless other factors are present. All toe associated buttons 614a-d, 615 are oblong in shape and extend toward the pedal center to reduce the number of foot controller sizes needed to accommodate users of different foot size. There is also a heel activated button 616 at the base of the pedal that may also be configured for the function it serves in brain function targeted games. All of the previously mentioned features also allow progress made during use of the controller over time to be monitored, recorded and analyzed. Useful comparison may then be made between the progress made using other methods of rehabilitation or brain function enhancement such that changes to enhance the eventual outcome for user and the game program/exercise program possibly amended to benefit all subsequent similar users.

Referring to FIG. 6B, 630 is a side view of the foot-actuated controller with certain internal components visible. Shown are enclosure 611 and foot pedal 612, which is connected near its base such that it can pivot and near its far end to a variable pressure producing sensor 631 such that sensor 631 may be configured to produce no back pressure, to produce free movement and where pedal position is entirely determined by user foot positioning; light to moderate, but constant, back pressure such that reduction in foot pressure on the part of the user causes return of the pedal to its extended position (pictured); and variable back pressure which increases as the pedal 612 is pushed further towards the base 611. The operational modes can be used both as rehabilitation monitoring tools and to configure the controller for most desirable function for brain function targeted game play. Also shown are a wireless network radio which may operate as part of a WIFI™, BLUETOOTH™ or ANT™ network systems 632. Data are delivered to a central controller processor 632 by data bus cables 634, 655. Power may be provided by a rechargeable power supply such as a battery or slow drain capacitor 633.

Referring to FIG. 6C, 650 is a detailed representation of foot pedal 612 according to the embodiment. Foot pedal 612 comprises a flat surface with a slight contoured indentation on the upward (outward facing) surface for comfortable receipt of a user's foot, which may be of one of a plurality of sizes. The end of foot pedal 612 meant to accept the user's heel has a single heel-activated button 616 which may be configured as a straight off-and-on switch or configured to sense changes in activation pressure 653 as needed for the conditions and needs of the brain function-targeted game to be played. The end of foot pedal 612 meant for the toes comprises an array of five buttons 615, 614a-d each positioned to correspond to one of the five toes 615, 614a, 614b, 614c, 614d. These buttons are highly configurable and can be programmed to act in unison as a single button (615-641a-d), or to act as two buttons, differentiating the large toe and the smaller toes (615, 614a-d). Last, for certain uses, having each toes register pressure independently 615, 614a, 614b, 614c, 614d may be desirable; this is a further configuration choice according to the embodiment. All data from foot pedal button actuators 651, 652a, 652b, 652c, 652d are sent to the enclosure 611 electronics (see FIG. 6B) through data cables 655.

FIGS. 7A-C comprise a set of diagrams illustrating aspects of an exemplary arrangement of a system for targeted neurogenesis augmented by aerobic exercise, illustrating the use of fixed or hand-held controllers and body positioning on a treadmill 700, as well as additional embodiment features of the treadmill 760, 770 according to a preferred embodiment of the invention. According to the embodiment, a user 714 may be standing, walking, or running on a treadmill or similar fitness device 700 with a stable base 710 and one or two separate moveable surfaces 711a, 711b for separate movement of the user's leg and fixed handlebars with affixed or integrally-formed controllers 511, 541 for use as connected smart devices for interaction, or support rails 718a, 718b for a user to hold onto for safety or interaction when needed. User 714 may interact with software applications using a variety of means, including manual interaction via controller devices 511, 541 that may be held in the hand or (as illustrated) may be affixed or integrally-formed into a treadmill 700. This may provide a user with traditional means of interacting with software applications while using treadmill 700. Additionally, a user's body position or movement may be tracked and used as input, for example via a plurality of tethers 716a-n affixed to handlebars 718a, 718b and a belt or harness 715 worn by user 714, or using a headset device 410 that may track the position or movement of a user's head. Body tracking may be used to recognize additional input data from user 714 (in addition to manual input via controllers 511, 541), by tracking the position and movement of user 714 during use. For example, motion tracking within a headset device 410 may be used to recognize a variety of translational 720 or rotational 730 movement of user's 714 head, such as leaning to the side, or looking over the shoulder. Tethers 716a-n may recognize a variety of movement of user's 714 torso, such as leaning, crouching, sidestepping, or other body movement. This body tracking may then be utilized as input similar to a control stick or joystick in manual controller arrangements, for example by interpreting the user's entire body as the “stick” and processing their body movements as if they were stick movements done manually. The tethers 716a-n, with minimal configuration changes, can be used to not only sense body movements but to act as body stabilizers by restricting their range of motion, allowing brain function targeted game participation to occur and user progress data to be collected, if planned, while maintaining needed safety margins.

For example, a user 714 on a treadmill 700 may be playing a virtual reality skiing game which incorporated brain function targeted tasks wherein they are given audio and video output via a headset 410 to immerse them in a virtual ski resort. While user 714 is skiing, they may be able to use manual controls 511, 514 for such operations as selecting from an on-screen icons or symbols, or typing finger pattern combinations or symbols integral to the brain function targeted task being performed. When they begin skiing within the game, user 714 may be instructed in proper posture or body movements such as shifting stance or picking up one foot or the other to improve balance during the ski experience, and may then use their body to control various aspects of their virtual skiing, such as leaning to the side 730 to alter their course and avoid trees or other skiers, or jumping 720 to clear rocks or gaps. Movement of their head may be detected by a headset 410 and used to control their view independently of their body as it is tracked by tethers 716a-n, allowing user 714 to look around freely without interfering with their other controls. In this manner, the user's entire body may serve as an input control device for the game, allowing and encouraging them to use natural body movements to control their gameplay in an immersive manner while still retaining the option to use more familiar manual control means as needed.

760 is a top-down view of a variable-resistance exercise treadmill machine 700 with wireless communication for smart device control and virtual reality applications, according to a preferred embodiment of the invention. According to the embodiment, treadmill 700 may comprise a stable base 710 to provide a platform for a user to safely stand or move about upon. Treadmill 700 may further comprise left 718a and right 718b hand rails for a user to brace against or grip during use, to provide a stable support for safety as well as a mounting point for external devices such as a plurality of tethers, as described above 716a-n. A plurality of steps 732a-n may be used to provide a user with a safe and easy means to approach or dismount treadmill 700, as well as a nonmoving “staging area” where a user may stand while they configure operation or wait for treadmill 700 to start operation. Unlike traditional treadmill machines common in the art, exercise machine 700 may be made with greater width to accommodate a wider range of free movement of a user's entire body (whereas traditional treadmills are designed to best accommodate only a jogging or running posture, with minimal lateral motion), and a plurality of separate moving surfaces 711a-b may be utilized to provide multiple separate surfaces that may move and be controlled independently of one another during use. For example, a user may move each of their legs independently without resistance applied, with separate moving surfaces 711a-b moving freely underfoot as a user applies pressure during their movement. This may provide the illusion of movement to a user while in reality they remain stationary with respect to their surroundings. Another use may be multiple separate moving surfaces 711a-b, with separate speeds of movement or degrees of resistance, so that as a user moves about during use they may experience physical feedback in the form of changing speed or resistance, indicating where they are standing or in what direction they are moving (for example, to orient a user wearing a virtual reality headset, as described above. Moving surfaces 711a-b may be formed with a texture 731 to increase traction, which may improve user safety and stability during use as well as improve the operation of moving surfaces 711a-b for use in multidirectional movement (as the user's foot is less likely to slide across a surface rather than taking purchase and applying directional pressure to produce movement). Use of multiple, multidirectional moving surfaces 711a-b may also be used in various therapeutic or rehabilitation roles, for example to aid a user in developing balance or range of motion. For example, a user who is recovering from an injury or surgery (such as a joint repair or replacement surgery) may require regular physical therapy during recovery. Use of multidirectional moving surfaces 711a-b along with appropriate guidance from a rehabilitation specialist or physical therapist (or optionally a virtual, remote coach using a software application or a specially designed game that targets specific physical weaknesses and reports progress) may make regular therapy more convenient and accessible to the user, rather than requiring in-home care or regular visits to a clinic. For example, by enabling a therapist or coach to manually vary the movement and resistance of the moving surfaces 711a-b, they can examine a user's ability to overcome resistance to different movements such as at odd angles or across varying range of motion, to examine the user's physical health or ability. By further varying the resistance it becomes possible to assist the user with rehabilitation by providing targeted resistance training to specific movements, positions, or muscle groups to assist in recovery and development of the user's abilities.

While FIG. 7 shows an embodiment of the invention comprising treadmill 700, it should be understood, and will be appreciated by one having ordinary skill in the art, that other common exercise machines may be used in accordance with the embodiment. For example, in a preferred embodiment, exercise machine 700 is an elliptical or similar running-oriented machine; generally, in augmented- or mixed-reality scenarios using exercise machines 700, it is of foremost importance to ensure the safety and comfort of participants (that is, participants should not be submitted to heightened risk of injury or disorientation). Treadmills may be problematic in this regard (although tether can alleviate this risk, as disclosed above), so in many cases neurogenesis-stimulating games will be provided in the contact of an elliptical machine 700. All examples provided herein may be used with treadmills, elliptical machines, or any other exercise equipment suitable for use in combination with various embodiments of the invention.

Treadmill 700 may be designed without a control interface commonly utilized by exercise machines in the art, instead being configured with any of a variety of wireless network interfaces such as Wi-Fi or BLUETOOTH™ for connection to a user's smart device, such as a smartphone or tablet computer. When connected, a user may use a software application on their device to configure or direct the operation of exercise machine 700, for example by manually configuring a variety of operation settings such as speed or resistance, or by interacting with a software application that automatically directs the operation of exercise machine 700 without exposing the particular details of operation to a user. Additionally, communication may be bi-directional, with a smart device directing the operation of exercise machine 700 and with exercise machine 700 providing input to a smart device based at least in part on a user's activity or interaction. For example, a user may interact with a game on their smart device, which directs the operation of exercise machine 700 during play as a form of interaction with, and feedback to, the user. For example, in a racing game, exercise machine 700 may alter the resistance of moving surfaces 711a-b as a user's speed changes within the game. In another example, a user may be moving about on moving surfaces 711a-b while playing a simulation or roleplaying game, and their movement may be provided to the connected smart device for use in controlling an in-game character's movement. Another example may be two-way interactive media control, wherein a user may select media such as music for listening on their smart device, and then while using exercise machine 700 their level of exertion (for example, the speed at which they run or jog) may be used to provide input to their smart device for controlling the playback of media. For example, if the user slows down music may be played slowly, distorting the audio unless the user increases their pace. In this manner, exercise machine 700 may be used interchangeably as a control and feedback device or both simultaneously, providing an immersive environment for a wide variety of software applications such as virtual reality, video games, fitness and health applications, or interactive media consumption.

770 is a side view of a variable-resistance exercise machine with wireless communication for smart device control and virtual reality applications, according to a preferred embodiment of the invention. According to the embodiment, an exercise machine 700 may have a stable base 710 to provide a platform for a user to safely stand or move about upon. Additional safety may be provided through the use of a plurality of integrally-formed or detachable side rails 718a, 718b, 736, for example having safety rails on the left and right sides (with respect to a user's point of view) of exercise machine 700 to provide a stable surface for a user to grasp as needed. Additionally, side rails 718a-b may comprise a plurality of open regions 735a-n formed to provide additional locations for a user to grasp or for the attachment of additional equipment such as a user's smart device (not shown) through the use of a mountable or clamping case or mount. Formed or removable supports 717a-n may be used for additional grip or mounting locations, for example to affix a plurality of tethers (not shown) for use in interaction with software applications while a user is using exercise machine 700.

Exercise machine 700 may further comprise a rigid handlebar 737 affixed or integrally-formed on one end of exercise machine 700, for a user to hold onto while facing forward during use. Handlebar 737 may further comprise a stand or mount 738 for a user's smart device such as (for example) a smartphone or tablet computer, so they may safely support and stow the device during use while keeping it readily accessible for interaction (for example, to configure or interact with a software application they are using, or to select different applications, or to control media playback during use, or other various uses). Handlebar 737 may be used to provide a stable handle for a user to hold onto during use for safety or stability, as well as providing a rigid point for the user to “push off” during use as needed, for example to begin using a moving treadmill surface 711a-b. During use, a user may also face away from handlebar 737, using exercise machine 700 in the reverse without their view or range of motion being obscured or obstructed by handlebar 737 (for example, for use with a virtual reality game that requires a wide degree of movement from the user's hands for interaction).

As illustrated, the base 710 of exercise machine may be formed with a mild, symmetrical curvature, to better approximate the natural range of movement of a user's body during use. Common exercise machines such as treadmills generally employ a flat surface, which can be uncomfortably during prolonged or vigorous use, and may cause complications with multi-directional movement or interaction while a user's view is obscured, as with a headset 410. By incorporating a gradual curvature, a user's movements may feel more natural and require less reorientation or accommodation to become fluid and proficient, and stress to the body may be reduced. A plurality of pressure sensors 739a-n may also be incorporated into the exercise machine's base 710 just below the treads 711a-b to allow monitoring of foot pick up at appropriate times during traditional rehabilitative sessions or sessions augmented by brain function targeted game play. This may be of particular importance to those suffering from neurodegenerative diseases such as Parkinson's disease where a shuffling gait often develops and even without the effects of performing brain function targeted tasks designed to ameliorate or reverse motor decline, careful attention to actions such as gait during walking has been found to prolong symptom onset, or certain stroke victims where attention to motor skills can significantly improve their recovery during the rehabilitation window. Such pressure sensors 739a-n also allow quantifiable measure or progress during particular physical action plans such as aerobic exercise combined with the performance of tasks targeted to specific brain functions.

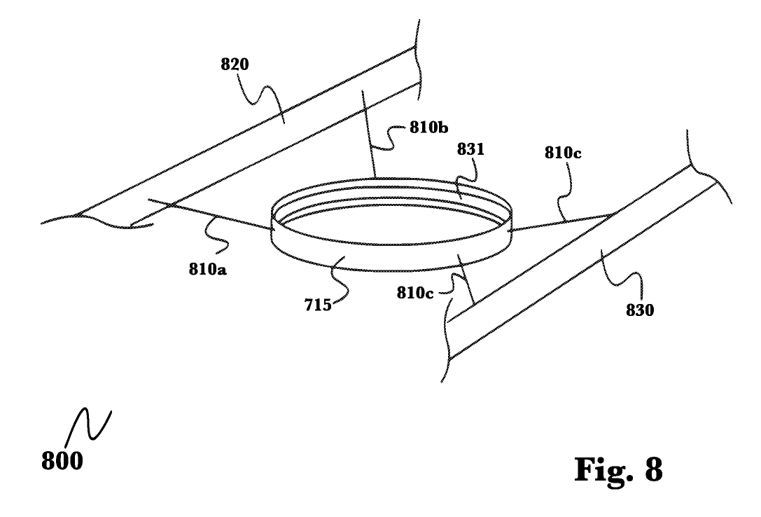

FIG. 8 is a diagram of an exemplary hardware arrangement of an apparatus for natural torso stabilization 800, tracking and feedback for electronic interaction illustrating the use of multiple tethers and a movable torso harness. According to the embodiment, a plurality of tethers 810a-n may be affixed or integrally-formed as part of a handle or railing 820, 830, such as handlebars found on exercise equipment such as a treadmill, elliptical trainer, stair-climbing machine, or the like. In alternate arrangements, specifically-designed equipment with integral tethers 810a-n may be used, but it may be appreciated that a modular design with tethers 810a-n that may be affixed and removed freely may be desirable for facilitating use with a variety of fitness equipment or structural elements of a building, according to a user's particular use case or circumstance. Tethers 810a-n may then be affixed or integrally-formed to a torso harness 715, as illustrated in the form of a belt, that may be worn by a user such that movement of their body affects tethers 810a-n and applies stress to them in a variety of manners. It should be appreciated that while a belt design for a torso harness 715 is shown for clarity, a variety of physical arrangements may be used such as including (but not limited to) a vest, a series of harness-like straps similar to climbing or rappelling equipment, a backpack, straps designed to be worn on a user's body underneath or in place of clothing (for example, for use in medical settings for collecting precise data) or a plurality of specially-formed clips or attachment points that may be readily affixed to a user's clothing. Additionally, a torso harness 715 may be constructed with movable parts, for example having an inner belt 831 that permits a user some degree of motion within the harness 715 without restricting their movement. Movement of inner belt 8311 (or other movable portions) may be measured in a variety of ways, such as using accelerometers, gyroscopes, or optical sensors, and this data may be used as interaction with software applications in addition to data collected from tethers 810a-n as described below.

As a user moves, their body naturally shifts position and orientation. These shifts may be detected and measured via tethers 810a-n, for example by detecting patterns of tension or strain on tethers 810a-n to indicate body orientation, or by measuring small changes in strain on tethers 810a-n to determine more precise movements such as body posture while a user is speaking, or specific characteristics of a user's stride or gait. Additionally, through varying the quantity and arrangement of tethers 810a-n, more precise or specialized forms of movement may be detected and measured (such as, for example, using a specific arrangement of multiple tethers connected to a particular area of a user's body to detect extremely small movements for medical diagnosis or fitness coaching). This data may be used as interaction with software applications, such as for virtual reality applications as input for a user to control a character in a brain function targeted game. In such an arrangement, when a user moves, this movement may be translated to an in-game character or avatar to convey a more natural sense of interaction and presence. For example, in a multiplayer roleplaying game, this may be used to facilitate nonverbal communication and recognition between players, as their distinct mannerisms and gestures may be conveyed in the game through detection of natural torso position and movement. In fitness or health applications, this data may be used to track and monitor a user's posture or ergonomic qualities, or to assist in coaching them for specific fitness activities such as holding a pose for yoga, stretching, or proper running form during use with a treadmill. In medical applications, this data may be used to assist in diagnosing injuries or deficiencies that may require attention, such as by detecting anomalies in movement or physiological adaptations to an unrecognized injury (such as when a user subconsciously shifts their weight off an injured foot or knee, without consciously realizing an issue is present).

Through various arrangements of tethers 810a-n and tether sensors (as described above, referring to FIG. 7), it may be possible to enable a variety of immersive ways for a user to interact with software applications, as well as to receive haptic feedback from applications. For example, by detecting rotation, tension, stress, or angle of tethers a user may interact with applications such as virtual reality games or simulations, by using natural body movements and positioning such as leaning, jumping, crouching, kneeling, turning, or shifting their weight in various directions to trigger actions within a software application configured to accept torso tracking input. By applying haptic feedback of varying form and intensity, applications may provide physical indication to a user of software events, such as applying tension to resist movement, pulling or tugging on a tether to move or “jerk” a user in a direction, or varying feedback to multiple tethers such as tugging and releasing in varying order or sequence to simulate more complex effects such as (for example, in a gaming use case) explosions, riding in a vehicle, or walking through foliage.

It should be appreciated that while reference is made to virtual reality applications, a wide variety of use cases may be possible according to the embodiment. For example, torso tracking may be used for fitness and health applications, to monitor a user's posture or gait while walking, without the use of additional virtual reality equipment or software. Configuration of secure limits to the range of motion of the tethers 810a-n may provide added stability and safety to those with balance or some lower extremity control deficiencies, especially if more substantial restraints are used than the waist band, allowing them to participate in certain brain function targeted task programs designed to improve those or perhaps unrelated deficiencies.

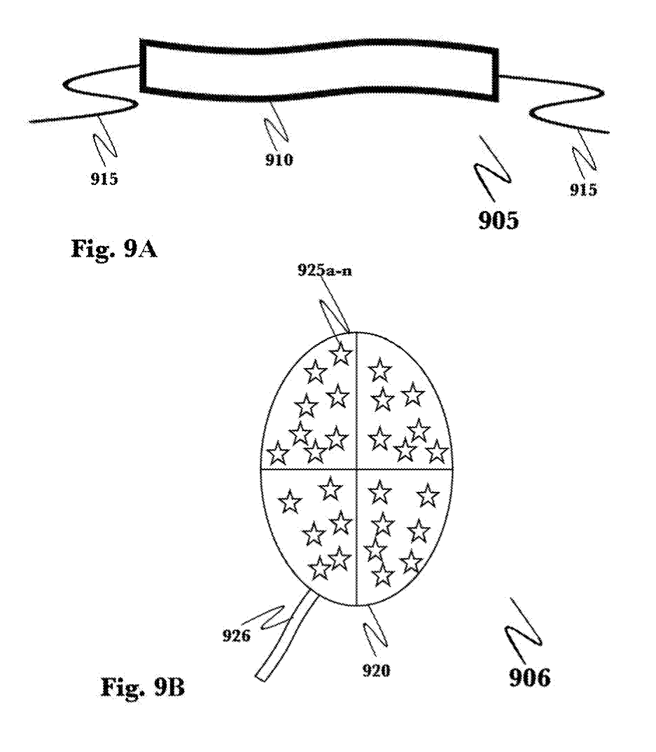

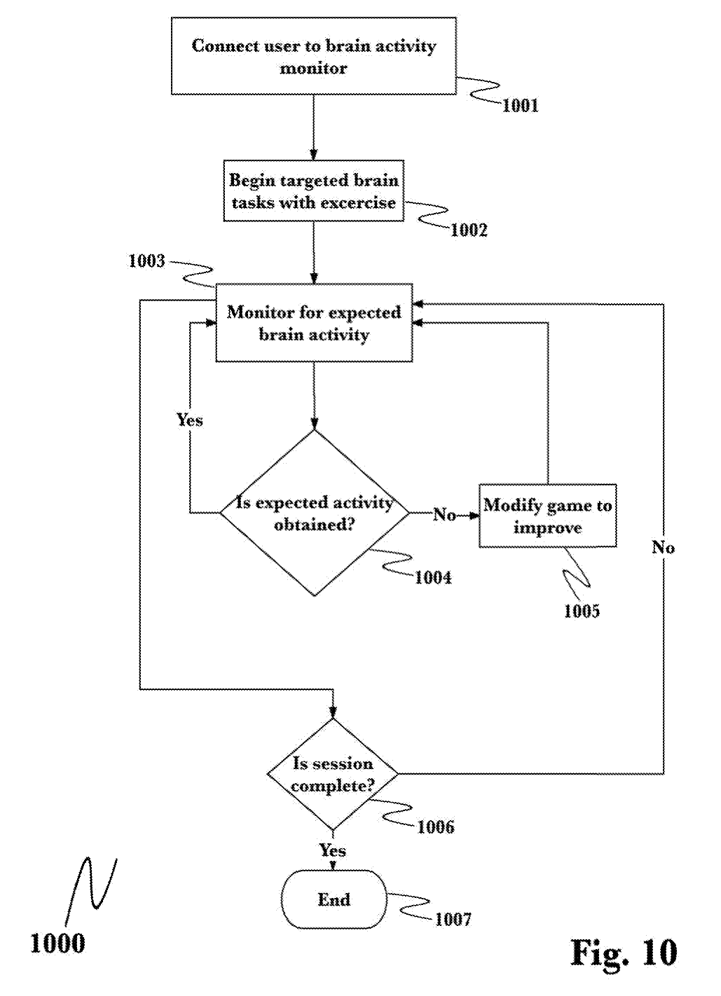

FIGS. 9A-9B show a set of diagrams illustrating physiological monitoring devices according to a preferred embodiment of the invention. During the course of use of games designed to improve certain targeted brain functions, especially when combined with aerobic exercise, one must collect physiological data to either confirm that a user is performing the two parts within the research set guidelines and to confirm administratively that expected physiological readings are present and thus the process may be working, the ultimate confirmation being actual improved brain function in the targeted areas. One tool is a set of sensors to measure exercise related parameter such as, but not necessarily limited to heart rate and respiration rate during the aerobic exercise that is part of the system. Research reports on aerobic correlated neurogenesis indicate that exertion within a certain range, which may differ somewhat depending on the brain function of interest, must be maintained for optimal outcome. Exercise that is too light or too strenuous reduce positive effects of exercise and keeping heart and respiration rates are used to monitor exertion levels. In the embodiment shown in FIG. 9A, a traditional chest band style heart-respiration rate monitor consisting of a central strap which encloses the sensors 910 and the lighter connector strap 915, which may facilitate respiration rate measurement is employed 905. Alternate embodiments might interface with other, third party devices that perform the same functions and are part of the user's normal routine such as, but not limited to FITBIT™, MICROSOFT BAND™, or APPLE WATCH™ devices. These devices might also be used, owing to their ability to record vital statistics, including heart rate, during workouts for later analysis, for users with pre-existing exercise regimens. FIG. 9B illustrates another possible indicator of efficacy related to targeted brain function related tasks is measurement of brain activity to confirm presence of anticipated patterns of brain wave activity. 906 shows a head cap type 920 sensor array 925a-n specifically designed to monitor the type of brain activity expected during use of the system. Sensor activity is carried for analysis by a conduit of cables 926. While this sensor array is expected to be used mainly in the programming stage of the brain function targeted task games, it may also be used in clinical settings to confirm correct function of the system with a wide range of users.

FIG. 10 is a flow diagram illustrating the use of brain monitoring equipment 1000 to evaluate efficacy of game-like targeted brain task programming according to a preferred embodiment of the invention. Users are first connected to the skull cap like sensor array 906 and baseline calibration is performed 1001. The user then begins both the neurogenesis enabling aerobic exercise regimen followed by the brain function targeting game under analysis 1002. Under some circumstances either the exercise regimen or the brain function targeting game may be performed alone while brain activity is monitored 1003 to establish whether effects are seen. During at least a portion of the session, brain activity is monitored 1003 both for comparison with that expected for activity in the region of the brain targeted by the game and for analysis at a later time. If observed brain activity 1004 is not as expected or is extremely weak, efforts may be made to improve the game through modification of the programmed actions 1005. Once the session is complete 1006, monitoring is ended 1007 and the user disconnected from the sensor array.

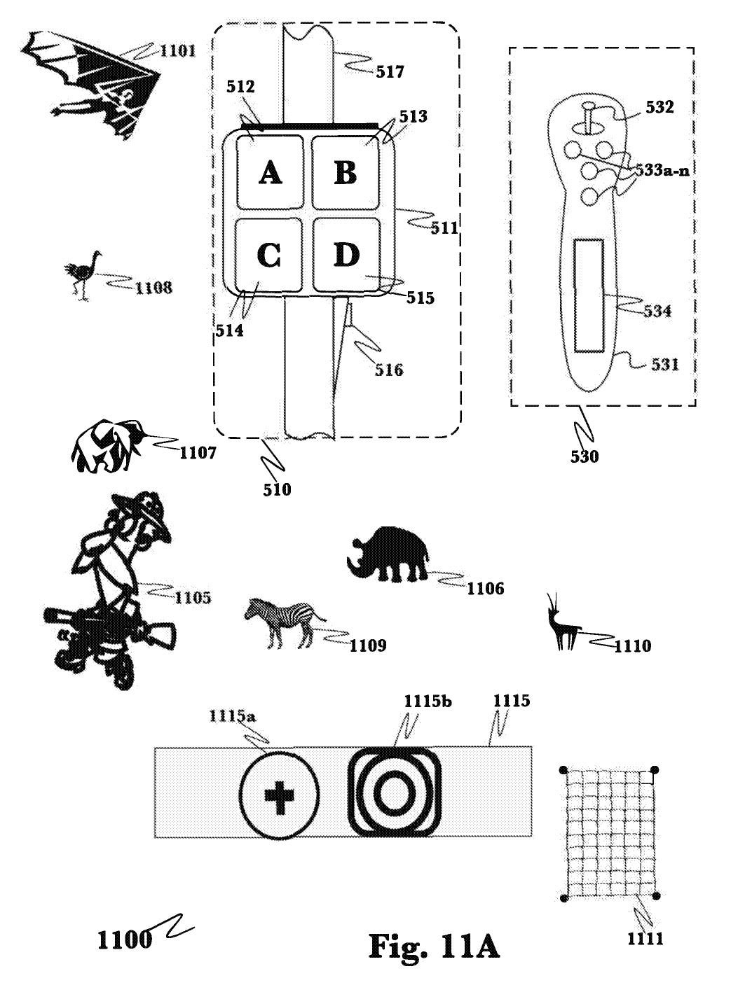

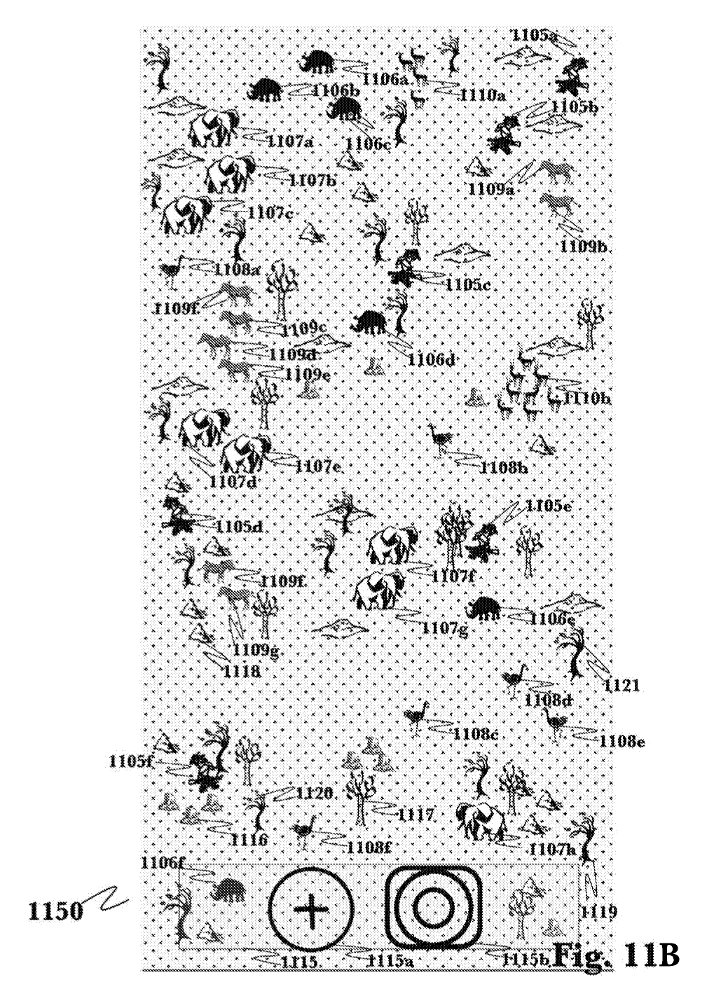

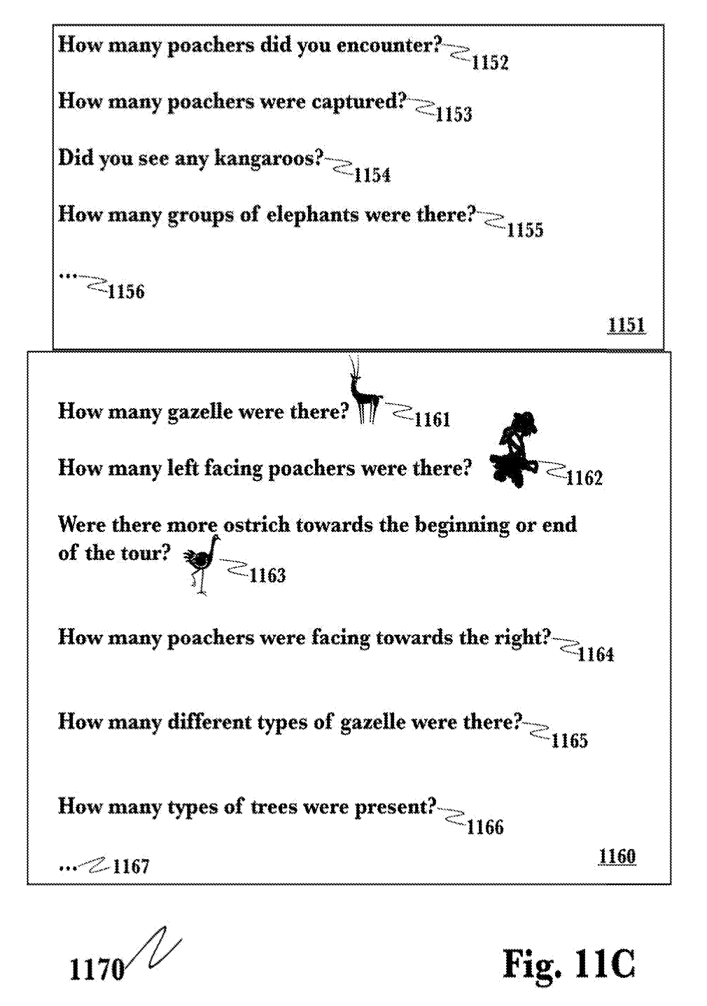

FIG. 11A shows a simplified rendering showing memory enhancing tasks presented in an aerobic-game format according to an embodiment of the invention. In FIG. 11A, 1100 depicts the parts that make up a fictitious African safari hang-gliding tour 1101 that may be played using one embodiment of the invention. During this game, a player may encounter several animals associated with an African safari from the vantage point of a hang glider traversing the African terrain. The height of the hang gliding experience may be controlled between three modes by the level of exertion of the player on the exercise machine coupled to the system which may be an elliptical in one embodiment or an exercise bike, ski machine, or specialized treadmill (see FIG. 7 and description, 700) in other embodiments, as measured by biometric sensors measuring heart rate, respiration rate (see 905) and possibly blood oxygen saturation such to keep exertion within published optimums for memory related neurogenesis. Too little exertion (resulting in vital statistics as sensed are below published optimal range) on the part of the player may cause the hang glider to descend and crash, in addition to a verbal warning; too much exertion (resulting in vital statistics above published optimal range) may cause the hang glider to climb and all items below to become blurry, in addition to a warning which may be visible or auditory depending on the embodiment. Exertion within the optimal range results in a level flight, pleasurable ride designed to fade into the background. Efficacy of the game to stimulate the players brain to enhance the desired function during play may optionally be monitored using a brain activity sensor cap or helmet as depicted in FIG. 9B, 906. The dual objects of the game are first to maneuver the path of flight to take pictures of the animals encountered during the tour 1106 to 1110 and second, when encountered, to maneuver the path of flight to capture poachers 1105 by launching nets onto them 1111. To assist in both of these game tasks, a high definition view area 1115 possessing aiming crosshairs 1115a for capturing the poachers and a photo reticle 1115b for capturing photos of the wildlife may be present. For this game one of multiple controllers may be used two of which may be the five button controller 510 that attaches to one of the elliptical poles 517 and where each of the four buttons forward buttons may be assigned a function including maneuvering to the left 512, maneuvering to the right 513, releasing 514 a poacher capturing net 1111, and releasing the camera shutter to photograph wildlife 515. Under other embodiments, the side button 516 may be assigned a role such as releasing the camera shutter, or there may be two five button controllers, one on each pole of the elliptical and the button roles divided between the two of them. Alternatively, an embodiment may use a controller similar to that depicted by 530 attached to a pole of the elliptical where a joystick 532 is used to move the flightpath to the left or right as the terrain moves under the player and buttons below the joystick used to activate release of the poacher capture net 1111, and activate the camera shutter. Again a second controller 510, attached to the second elliptical pole may be used to release the net 1111 and the camera shutter using two of its buttons (for example, 512, 513). Other embodiments may use the same controller configurations but use an exercise bike or specially designed treadmill as the exercise vehicle. Some embodiments may use a specially designed harness to sense player body movements FIG. 8, 800 to position the hang glider. For example, for a user requiring cognitive and memory sessions or treatments, the user may be tasked to do animal surveys. In this case, the user will be looking for specific animal (using cognitive skills), take photos of them, and counting them (using memory skills) as they glide over a safari. On the cognitive skills, the game will capture the user's response time from each moment the targeted animal shows up in their field of vision to the time they activate the camera, including scenarios where the user completely misses the animal. At the end of the session, the user may be asked to answer animal-specific questions for the short-term memory tests and factoid questions for the long-term memory tests. For a user requiring motor skill sessions (for example, because of neurodegenerative disease), the game will serve up more poachers during the animal survey and will capture the additional results whether the user can capture them or not and the time durations. The game can also serve up more obstacles for the user to navigate through, or other additional challenges and gameplays elements as needed.

Examples of some of the animals used in a plurality of the game instances, as the animal make-up and terrain, possibly containing obstacles to hang glider flight that must be dealt with, of each game may differ to assist in retaining higher interest and attention levels on the part of the user are: Rhinoceros 1106, elephant 1107, ostrich 1108, gazelle 1110 and zebra 1109. Rendition of all constituent parts of the game may be realistic for each of those parts, the icon-like representation here is for illustration purposes only.

Referring to FIG. 11B, 1150 shows what a short segment from a run of the game may look like, greatly simplified for clarity of illustration. The high definition area of vision is shown at the bottom of the FIG. 1115, with the sight for poacher capture sight 1115a and camera reticle 1115b for photographing wildlife encountered. While the player may see a distance beyond this area for maneuver planning, this area 1115 is the area of clearest view. Multiple poachers 1105a, 1105b, 1105c, 1105d, 1105e, 1105f are present as are several rhinoceros 1106a, 1106b, 1106c, 1106d, 1106e, 1106f, elephants 1107a, 1107b, 1107c, 1107d, 1107e, 1107f, 1107g, 1107h, ostrich 1108a, 1108b, 1108c, 1108d, 1108e, 1108f, zebra 1109a, 1109b, 1109c, 1109d, 1109e, 1109f, 1109g and two herds of gazelle 1110a, 1110b, found in small groups 1109c, 1109d, 1109e, 1109f, 1107a, 1107b, 1107c; herds 1110a, 1110b; or individually 1108b, 1106d, 1108c. There may also be a number of trees of different types 1117, 1119, 1120, 1121 and rocks 1116, 1118. Such information may become important in the next phase of the game when the player may be asked to recall such facts as part of memory enhancement exercises (see FIG. 11C). As stated, the game field shown may be only a small section of the area encompassed by an actual game run, in fact one method of increasing the difficulty of the memory exercise task underlying the game play may be to elongate the amount of time the player spends touring the African terrain.

Referring to FIG. 11C, 1170 introduces some sample questions that a player may be asked to answer after a hang glider tour. Depending on the level of ability of the player and her experience (level) with the game, the questions posed may differ greatly from easy 1151, where, among others the player may be asked how many poachers they encountered 1152 as the system keeps track of all objects passing through the HD visibility zone 1115, which, by instruction may be what the player is responsible for remembering; how many poachers she was able to capture 1153; if any kangaroos were seen 1154 (obviously they would be out of place in a realistic safari scenario, but are exemplary of game mechanics; any animals could be used in place of any others of course); how many groups of elephants were seen 1155; among other possible questions at this difficulty level 1156. After a short acquaintance building period for some or a build of proficiency due to game play and targeted brain function enhancement through exercise and memory challenge, many players may increase the difficulty level of what aspects of the game mediated tour they must be able to recall through encounter with significantly more difficult questions 1160. Such questions, pulled from the presented, simplified, game activity may include: How many gazelle were encountered 1161; How many left facing poachers were there 1162; Were there more ostrich towards the beginning of the tour or more towards the end of the tour 1163; How many poachers were facing towards the right 1164; How many different types of gazelle were present 1165; and How many types of trees were present 1166; just to list a small number of examples from the plurality of possible memory related queries imaginable by those skilled in the art 1167. The use of colored, complex, more realistic graphics with shading in the actual production game greatly increases what can be done to both keep the game play more interesting and greatly adds to what can be done during the memory recall portion of the brain function enhancement task.

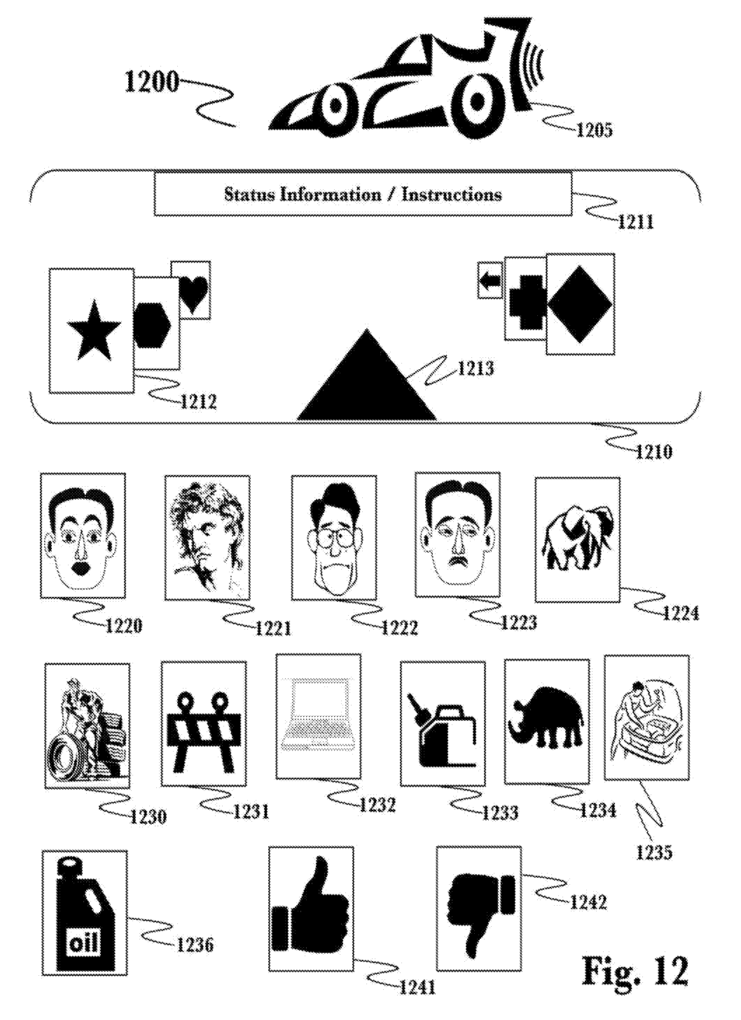

FIG. 12 is a set of simplified illustrations showing a generalized aerobic exercise brain function enhancement engine which may be used for multiple brain function enhancement tasks 1200. A second aerobic-exercise enhanced brain function enhancement game is depicted here as a first person racing game but the principle and props used in the engine could take the form of a running or bicycling tour through scenery of interest while performing the requisite tasks aimed to direct enhanced function of desired brain functions. Using the racing embodiment, the player would assume the role of driver of a racecar 1205. The field of view in the AR/VR viewer display 1210 may comprise an area where car status and in game instructions such as, but not limited to, what to search for and collect are displayed 1211, an indicator of the front end of the car (or front end of the bicycle or runner's hands in other possible embodiments) 1213 and a series of items, shown as icons floating in rectangular placards 1212 here for ease of illustration, situated along the path of travel of the race car such that the player can steer to either hit and collect them or drive to avoid them depending on the objects of the particular game run. The player may, as described in FIG. 11A, control the forward motion of the car in a simple fashion by maintaining a level of exertion, as measured, for example, by attached biometric sensors for heart rate, respiration rate and, in some embodiments, blood oxygenation level, found to be optimal for neurogenesis-based effects on the particular brain function that is the target of the tasks presented in the game. Optimal range exertion may maintain car speed at maximal safe levels, too little exertion may cause the car to slow to where other cars in the race begin to pass the player's car 1213 and a warning to appear in the status display 1211 whereas too much exertion may cause the player's car 1213 to overheat and slow or some similar ill-effect again causing reduced car function and present a warning in the display indicating that exercise exertion has risen above target levels and a modest reduction is required 1211. Without player controller input, the race car will continue in a straight course on the track. Lateral motion, left or right may be accomplished by depressing a button in a predesignated set of buttons, usually one button for left and another for right on one of the previously described controllers such as FIG. 5, 510, a controller joystick such as the one described in FIG. 5, 530, 532 may also be used. These controllers may be mounted to one or both handles of an elliptical type exercise machine, or an exercise bike or specially designed treadmill FIG. 7 according to the embodiment.

People with autism spectrum disorder (ASD) suffer from many debilitating mental challenges which, among other symptoms, presents as significant to severe social interaction deficiencies which impact their ability to interact with other people during years at school and retain employment later in life. A highly significant portion of the social issues arise from the apparent inability of people throughout the autism spectrum to recognize other people's emotions or intentions from facial queues and other non-verbal indicators that are naturally recognized by the normal population. Physiologically, the structures of the brain believed responsible include the amygdala and more recently, it has been proposed that visible reduction in the size of the amygdala, which acts as a conduit into the other portions of the brain cited as deficiencies in autism, may be the controlling moderator in autism severity and symptoms. The amygdala has long been shown to directly benefit from the neurogenesis effect of aerobic exercise and it therefore stands to reason that people with ASD may benefit from targeted brain function enhancement coupled with aerobic exercise. One embodiment of a puzzle game where a group of players must collaborate and peddle together to solve the game puzzle, they will need help from certain game characters, and will be rewarded or challenged by their different facial emotions or expressions. ASD encompasses a wide spectrum of abilities in this area, so the starting point of the treatment may also vary greatly, from starting out with extremely simplified facial representations depicting only the important facial features in emotion prediction which, from study of the general population, have been found to be the eye region including the lids and eyebrows and the mouth region as shown in 1220, 1223 and the slightly more complex image depicted in 1222 (glasses). The player may be tasked with finding faces of subjects who are “sad,” which the face in 1223 is a simple example and then presented with a plurality of images on the race track some of which express simple or possible variations on sadness in facial expressions. Under the normal play of the game, correct choices may cause the car to speed up relative to opponents. Under the conditions of ASD other rewards, such as promised time to discuss the ASD individual's area of hyperfocus, may need to be offered to maintain focus and interest. The game could possibly be modified to make use of the individual's area of hyperfocus to significantly enhance mental commitment. For example, if the player's area of hyperfocus is horror movies and props, the game could instead be a tour of an endless grave yard or the like. As the player becomes more advanced in facial recognition, the goal may be to present her with photorealistic facial examples, represented simplistically by 1221 and also present variants in facial features for emotions to present the most realistic environment. Many irrelevant images 1224 may be intermixed with faces. The use of audible instructions may also be desirable in certain cases.

Like facial recognition, people with ASD may have extreme difficulty determining intent of others during conversation and have significant difficulty attaching immediacy to instructions or showing judgement with questionable directives. Using audio clips of a wide variety of phrases and requests using a plurality of delivery intonations and then either “Yes” (“Good”) 1241 or “No” (“BAD”) 1242 indicators within the game, this are of underachievement may be addressed. For example, the player my first be presented with a request from a “friend” to do something the player knows is wrong with the speaker indicating that a friend would do such an act for his friends and then asked whether the act should be done or not or whether the speaker is a friend or not. The player may then indicate her answer by choosing “Yes” 1241 or “No” 1242. Again, irrelevant choices 1224 may be offered to indicate focus and game efficacy. The game would then be made up of a plurality of spoken passages having to do with time immediacy or meaning of the request when rephrased to exercise these needed skills.

Another example brain function exercising game may provide tasks to exercise executive thinking where one must decide whether a presented item would have a positive effect or negative effect on a stated goal. Given a stated goal of maintaining a car in top working order a game may present a plurality of images 1230, 1231, 1232, 1233, 1234, 1235, 1236 which may assist in the goal 1230, 1233, 1235, 1236, detract from the goal 1234 or have no relevance to the stated goal 1231. The items may have close relation to the theme of the game 1231 but no effect on the goal and images for items that are marginally positive or negative to the goal may be presented, in this case, such as but not limited to a car wash (not shown).

The examples above represent a very small sampling of the tasks and themes that may be presented by the brain function enhancement gaming engine 1200. For clarity, illustrations are also extremely simplistic compared to the capabilities of the engine to display photorealistic objects and scenery. The examples given in no way should be taken to limit the engine's ability to present widely varied images and information or to limit the areas of brain function exercise available through use of the engine, which is designed to provide tasks, both visual and auditory to exercise the function of any brain area identified as beneficial to subject ability and accessible to such targeted engagement known to those skilled in the art.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

Exemplary virtual reality hardware or devices used in description above may include Sony's PLAYSTATION VR™ console equipped with a VR headset, an OCULUS RIFT™ VR headset, Microsoft's XBOX ONE™ VR console with VR headset, or a headset developed specifically for this purpose.

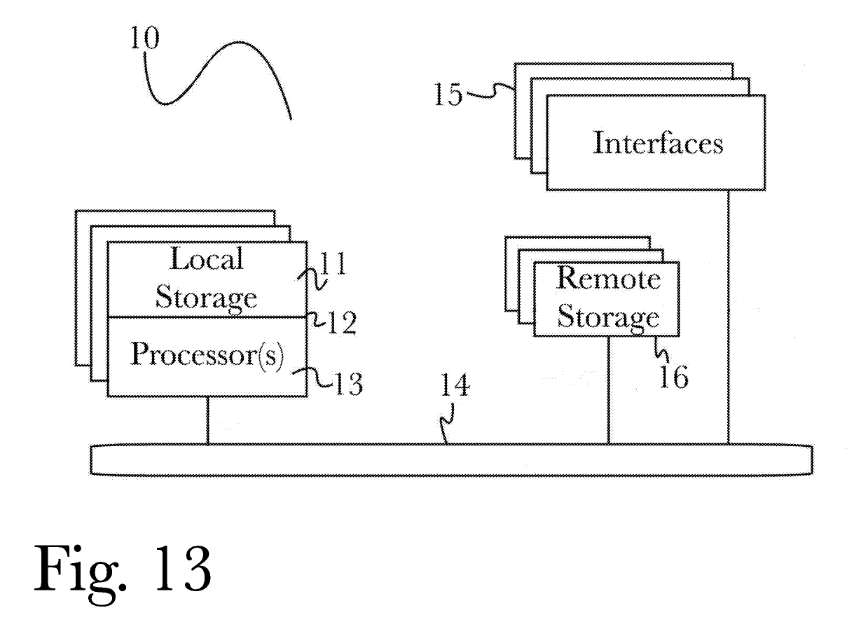

Referring now to FIG. 13, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one embodiment, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one embodiment, a computing device 10 may be configured or designed to function as a server system utilizing CPU 12, local memory 11 and/or remote memory 16, and interface(s) 15. In at least one embodiment, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a specific embodiment, a local memory 11 (such as non-volatile random access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a Qualcomm SNAPDRAGON™ or Samsung EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one embodiment, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 13 illustrates one specific architecture for a computing device 10 for implementing one or more of the inventions described herein, it is by no means the only device architecture on which at least a portion of the features and techniques described herein may be implemented. For example, architectures having one or any number of processors 13 may be used, and such processors 13 may be present in a single device or distributed among any number of devices. In one embodiment, a single processor 13 handles communications as well as routing computations, while in other embodiments a separate dedicated communications processor may be provided. In various embodiments, different types of features or functionalities may be implemented in a system according to the invention that includes a client device (such as a tablet device or smartphone running client software) and server systems (such as a server system described in more detail below).

Regardless of network device configuration, the system of the present invention may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

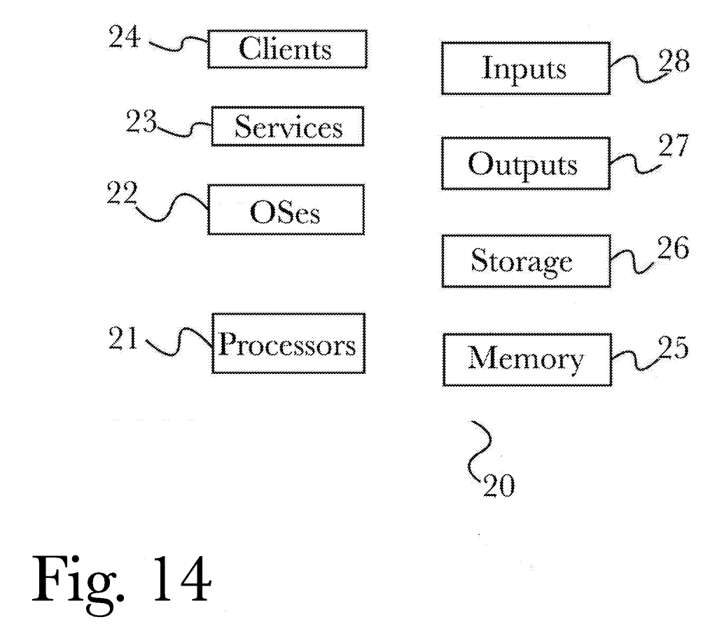

In some embodiments, systems according to the present invention may be implemented on a standalone computing system. Referring now to FIG. 14, there is shown a block diagram depicting a typical exemplary architecture of one or more embodiments or components thereof on a standalone computing system. Computing device 20 includes processors 21 that may run software that carry out one or more functions or applications of embodiments of the invention, such as for example a client application 24. Processors 21 may carry out computing instructions under control of an operating system 22 such as, for example, a version of Microsoft's WINDOWS™ operating system, Apple's Mac OS/X or iOS operating systems, some variety of the Linux operating system, Google's ANDROID™ operating system, or the like. In many cases, one or more shared services 23 may be operable in system 20, and may be useful for providing common services to client applications 24. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 13). Examples of storage devices 26 include flash memory, magnetic hard drive, CD-ROM, and/or the like.

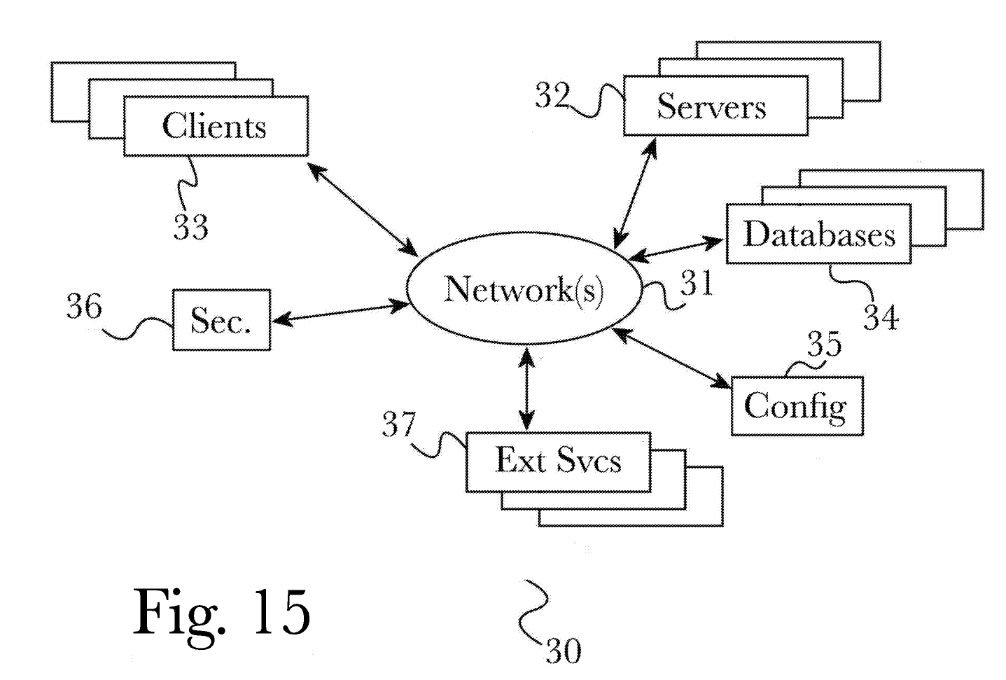

In some embodiments, systems of the present invention may be implemented on a distributed computing network, such as one having any number of clients and/or servers. Referring now to FIG. 15, there is shown a block diagram depicting an exemplary architecture 30 for implementing at least a portion of a system according to an embodiment of the invention on a distributed computing network. According to the embodiment, any number of clients 33 may be provided. Each client 33 may run software for implementing client-side portions of the present invention; clients may comprise a system 20 such as that illustrated in FIG. 13. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as Wi-Fi, WiMAX™, LTE, and so forth), or a local area network (or indeed any network topology known in the art; the invention does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in an embodiment where client applications 24 are implemented on a smartphone or other electronic device, client applications 24 may obtain information stored in a server system 32 in the cloud or on an external service 37 deployed on one or more of a particular enterprise's or user's premises.

In some embodiments of the invention, clients 33 or servers 32 (or both) may make use of one or more specialized services or appliances that may be deployed locally or remotely across one or more networks 31. For example, one or more databases 34 may be used or referred to by one or more embodiments of the invention. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, Hadoop Cassandra, Google BIGTABLE™, and so forth). In some embodiments, variant database architectures such as column-oriented databases, in-memory databases, clustered databases, distributed databases, or even flat file data repositories may be used according to the invention. It will be appreciated by one having ordinary skill in the art that any combination of known or future database technologies may be used as appropriate, unless a specific database technology or a specific arrangement of components is specified for a particular embodiment herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, most embodiments of the invention may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments of the invention without limitation, unless a specific security 36 or configuration system 35 or approach is specifically required by the description of any specific embodiment.

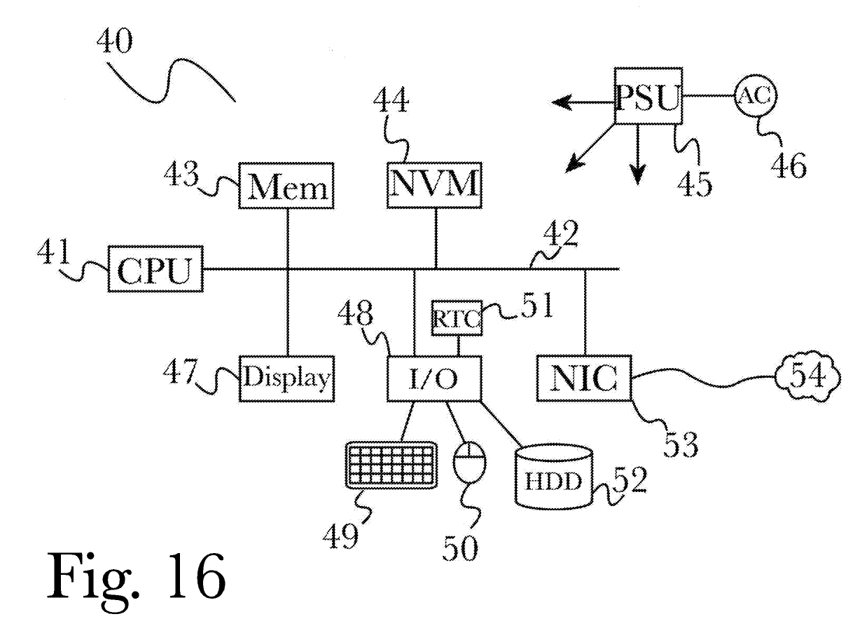

FIG. 16 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of the present invention may be distributed among any number of client and/or server components. For example, various software modules may be implemented for performing various functions in connection with the present invention, and such modules may be variously implemented to run on server and/or client components.

The skilled person will be aware of a range of possible modifications of the various embodiments described above. Accordingly, the present invention is defined by the claims and their equivalents.

What is claimed is:

1. A system for targeted neurogenesis stimulated by aerobic exercise with brain function-specific tasks, comprising:
a game server operating on a computing device comprising at least a processor and a memory and configured to:
operate a game-oriented software program comprising game data configured to stimulate at least a specific brain function in a user during gameplay;
transmit at least a portion of the game data to a virtual reality device;
receive user input from a plurality of devices via a network; and
direct the operation of the game-oriented software program based at least in part on the user input; and
a virtual reality device comprising at least a processor and a memory and a plurality of hardware sensors and a display device, and configured to:
receive game data from the game server;
display at least a portion of the game data to a user using the display device;
transmit operating instructions to a plurality of external devices, the instructions being based at least in part on at least a portion of the game data;
receive input from the user via at least a portion of the plurality of hardware sensors; and
transmit at least a portion of the user input to the game server.

2. The system of claim 1, wherein the game server is further configured to:
receive biometric data from a plurality of fitness devices; and
direct the operation of the game-oriented software program based at least in part on the received biometric data.

3. The system of claim 2, wherein the biometric data comprises heart rate data.

4. The system of claim 2, wherein the biometric data comprises blood oxygenation data.

5. The system of claim 2, wherein the biometric data comprises accelerometer data.

6. A method for targeted neurogenesis stimulated by aerobic exercise with brain function-specific tasks, comprising the steps of:
operating, using a game server operating on a computing device comprising at least a processor and a memory, a game-oriented software program comprising game data configured to stimulate at least a specific brain function in a user during gameplay;
transmitting at least a portion of the game data to a virtual reality device;
receiving, at a virtual reality device comprising at least a processor and a memory and a plurality of hardware sensors and a display device, game data from the game server;
displaying at least a portion of the game data to a user using the display device;
receiving input from the user via at least a portion of the plurality of hardware sensors;
transmitting at least a portion of the user input to the game server; and
directing the operation of the game-oriented software program based at least in part on the user input.

7. The method of claim 6, further comprising the steps of:
receiving biometric data from a plurality of fitness devices; and
directing the operation of the game-oriented software program based at least in part on the received biometric data.

8. The method of claim 7, wherein the biometric data comprises heart rate data.

9. The method of claim 7, wherein the biometric data comprises blood oxygenation data.

10. The method of claim 7, wherein the biometric data comprises accelerometer data.
